(19) United States
(12) Patent Application Publication
(10) Pub. No.: US 2022/0371989 A1
(43) Pub. Date: Nov. 24, 2022

(54) N-ACYL-TYROSINE DERIVATIVES AND USES THEREOF

(71) Applicant: Sederma, Le Perray en Yvelines (FR)

(72) Inventors: Logan William MacIntyre, Charlottetown (CA); Russell Greig Kerr, Charlottetown (CA); Bradley Haltli, New Haven (CA)

(73) Assignee: Sederma, Le Perray en Yvelines (FR)

(21) Appl. No.: 17/642,581

(22) PCT Filed: Sep. 11, 2020
(86) PCT No.: PCT/CA2020/051228

§ 371 Date: Mar. 11, 2022

Related U.S. Application Data
(60) Provisional application No. 62/900,210, filed on Sep. 13, 2019.

(51) Int. Cl. C07C 233/47; A61P 31/04; C12N 1/20; C12P 13/22

(52) U.S. Cl. CPC C07C 233/47; A61P 31/04; C12N 1/20; C12N 1/205; C12P 13/225; C12R 2001/01

(57) ABSTRACT

Provided herein are compounds of Formula A, methods for the preparation thereof, and uses thereof for treating or preventing bacterial infections.

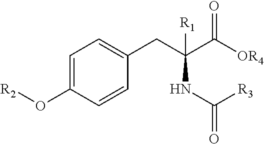

### CROSS-REFERENCE TO RELATED APPLICATIONS

The present application claims priority to U.S. provisional patent application Ser. No. 62/900,210, filed on Sep. 13, 2019, the contents of which are incorporated herein by reference in their entirety.

### DEPOSIT OF BIOLOGICAL MATERIAL

Alteromonas sp. RKMC-009 has been deposited with the Agricultural Research Culture Collection (NRRL) International Depositary Authority on Aug. 17, 2020 (the assigned “date of the original deposit”), and assigned Accession Number NRRL B-67979. The deposit was made under the provisions of the Budapest Treaty on the International Recognition of the Deposit of Microorganisms for the Purpose of Patent Procedures. Copies of the “Receipt in the Case of an Original Deposit” and the “Viability Statement” issued by the IDA, as well as FORM PCT/RO/134, are included below.

### FIELD OF INVENTION

The present invention relates generally to tyrosine derivatives. More specifically, the present invention relates to N-acyl, α-alkyl tyrosine derivatives and uses thereof.

### BACKGROUND

Cultured environmental bacteria may produce a plethora of antimicrobial natural products with diverse chemical structures; however, culture-independent studies indicate that only a small fraction of bacterial species from most habitats have been cultured. The ichip is a device that may allow many members of the “uncultured majority” to be cultured in the laboratory, thereby facilitating antimicrobial discovery.

It is becoming increasingly apparent that microbial natural product discovery is hampered by an inability to culture most environmental bacteria using traditional cultivation techniques. (1, 2) One particular approach for accessing the “uncultured majority” may employ diffusion chambers for in situ cultivation. Bacterial inoculum from a given habitat may be housed within diffusion chambers that may then be incubated in their natural environments until the resulting colonies have reached a sufficient size for “domestication” in the laboratory; it is presumed that this process may be mediated by diffusible growth factors from the environment. (3) Diffusion chamber technology was made more compatible with high-throughput natural product discovery by the development of the ichip (isolation chip). (4) This device contains hundreds of miniaturized diffusion chambers that are inoculated with a single environmental bacterial cell thereby allowing concomitant in situ incubation and isolation. The ichip has been shown to both enable the cultivation of a greater proportion of environmental bacteria and afford greater taxonomic novelty among recovered isolates. The utility of the ichip in bacterial natural product discovery was demonstrated by the isolation of teixobactin, which is a promising antibacterial for clinical use and produced by a previously unreported species of soil bacterium (Eleftheria terrae). (5)

Environmental bacterial and other microbes may produce a wide variety of useful compounds which have not yet been discovered. Given the increasing problem of antibiotic resistance in several important bacterial infections affecting humans and other animals, the identification of new antibiotic compounds is highly desirable in the field.

Alternative, additional, and/or improved compounds having desirable effects such as antibacterial properties are desirable.

### SUMMARY OF INVENTION

As described in detail herein, an N-acyl-α-methyl tyrosine derivative has been discovered through isolation from a Alteromonas sp. RKMC-009 bacterium obtained from the marine sponge Xestospongia muta. The antibacterial activity and structure-activity relationships (SAR) of the obtained N-acyl-tyrosine derivative have been extensively studied, and additional synthetically prepared analogues or derivatives have been prepared also featuring antibacterial activity. Synthetic methods for preparing such compounds, and uses thereof in the treatment or prevention of bacterial infection, are also described.

In an embodiment, there is provided herein a compound of formula A:

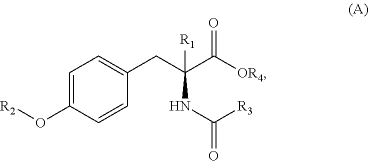

wherein
   R₁ is —H; or a linear or branched, optionally substituted, C₁-C₆ alkyl; or optionally substituted phenyl;
   R₂ is —H; or a linear or branched, optionally substituted, C₁-C₆ alkyl; or optionally substituted phenyl;
   R₃ is linear or branched, optionally substituted, C₅-C₂₀ alkyl, alkenyl, or alkynyl; and
   R₄ is —H; linear or branched, optionally substituted, C₁-C₆ alkyl; or optionally substituted phenyl;
or a prodrug, ester, or pharmaceutically acceptable salt or solvate thereof.

In another embodiment of the above compound, R₁ may be —H, Me, Et, nPr, iPr, tBu, iBu, secBu, nBu, or Ph.

In still another embodiment of any of the above compound or compounds, R₂ may be —H, Me, Et, nPr, iPr, tBu, iBu, secBu, nBu, or Ph.

In yet another embodiment of any of the above compound or compounds, R₃ may be a C₅-C₂₀ group with 0-3 double and/or triple carbon-carbon bonds (0-3 Δ).

In another embodiment of any of the above compound or compounds, R₃ may be a linear C₅-C₂₀ group with 0-3 double and/or triple carbon-carbon bonds (0-3 Δ).

In yet another embodiment of any of the above compound or compounds, R₄ may be —H, Me, Et, or Ph.

In still another embodiment of any of the above compound or compounds, the compound may be a compound having formula B:

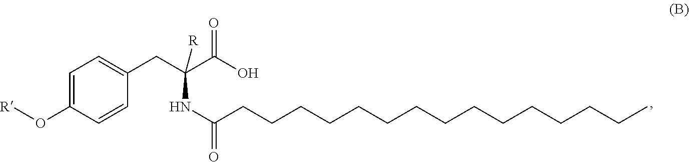

wherein
   R is —CH₃ or —H; and
   R′ is —CH₃ or —H;
or a prodrug, ester, or pharmaceutically acceptable salt or solvate thereof.

In another embodiment of any of the above compound or compounds, the compound may be a compound having formula 1:

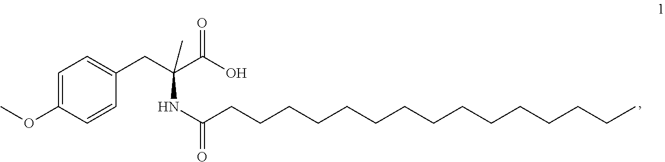 [SMILES: CCCCCCCCCCCCCCCC(=O)N[C@](C)(Cc1ccc(OC)cc1)C(=O)O]

or a prodrug, ester, or pharmaceutically acceptable salt or solvate thereof.

In another embodiment of any of the above compound or compounds, the compound may be a compound having formula 2:

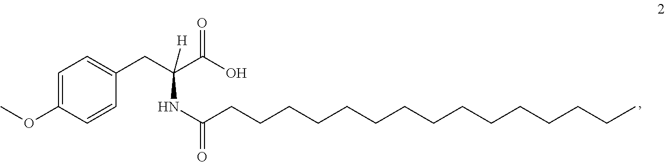 [SMILES: CCCCCCCCCCCCCCCC(=O)N[C@H](Cc1ccc(OC)cc1)C(=O)O]

or a prodrug, ester, or pharmaceutically acceptable salt or solvate thereof.

In another embodiment of any of the above compound or compounds, the compound may be a compound having formula 3:

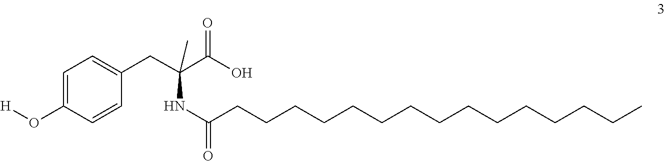 [SMILES: CCCCCCCCCCCCCCCC(=O)N[C@](C)(Cc1ccc(O)cc1)C(=O)O]

or a prodrug, ester, or pharmaceutically acceptable salt or solvate thereof.

In another embodiment of any of the above compound or compounds, the compound may be a compound having formula 4:

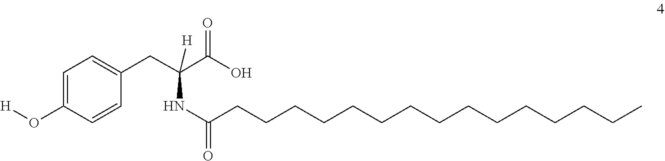 [SMILES: CCCCCCCCCCCCCCCC(=O)N[C@H](Cc1ccc(O)cc1)C(=O)O]

or a prodrug, ester, or pharmaceutically acceptable salt or solvate thereof.

In another embodiment, there is provided herein a pharmaceutical composition comprising any one or more of the compound or compounds as described herein, and a pharmaceutically acceptable excipient or carrier. The compound as described herein may be synthetically produced and/or in isolated form.

In yet another embodiment, there is provided herein a use of any of the compound or compounds as described herein, or any of the pharmaceutical composition or compositions as described herein, as an antibacterial agent, or in the manufacture of an antibacterial medicament.

In another embodiment, there is provided herein a use any of the compound or compounds as described herein, or any of the pharmaceutical composition or compositions as described herein, for treating a bacterial infection.

In another embodiment, there is provided herein a method for reducing or preventing growth of a bacteria, said method comprising:
contacting the bacteria with any of the compound or compounds as described herein, or any of the pharmaceutical composition or compositions as described herein.

In still another embodiment, there is provided herein a method for treating or preventing a bacterial infection in a subject in need thereof, said method comprising:
administering any of the compound or compounds as described herein, or any of the pharmaceutical composition or compositions as described herein, to the subject.

In certain embodiments, the bacteria may be or comprise a Staphylococcus or Enterococcus bacteria. In another embodiment, the bacteria may be or comprise Staphylococcus aureus, Staphylococcus warneri, or Enterococcus faecium. In another embodiment, the bacteria may be or comprise methicillin-resistant Staphylococcus aureus (MRSA), or vancomycin-resistant Enterococcus faecium (VRE). In still another embodiment, the bacteria may be or comprise E. faecium EF379 (VRE), E. faecium 15337, E. faecalis 16371, E. gallinarum 20993, E. casselihflavus 15984, E. hirae 17446, S. aureus ATCC 33591 (MRSA), or S. warneri ATCC 17917.

In another embodiment of any of the above uses or methods, the bacteria may comprise an Enterococcus and the compound may comprise formula 1

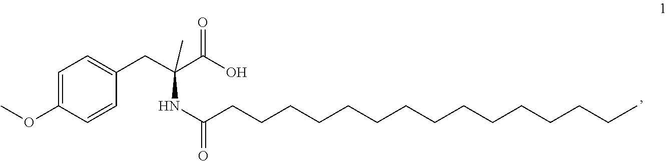 [SMILES: CCCCCCCCCCCCCCCC(=O)N[C@](C)(Cc1ccc(OC)cc1)C(=O)O]

or a prodrug, ester, or pharmaceutically acceptable salt or solvate thereof.

In another embodiment of any of the above uses or methods, the bacteria may comprise an Enterococcus and the compound may comprise formula 3

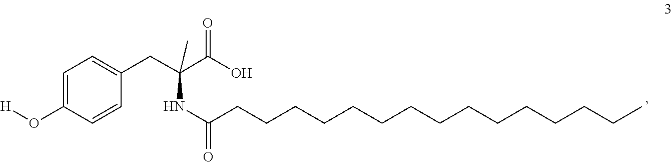 [SMILES: CCCCCCCCCCCCCCCC(=O)N[C@](C)(Cc1ccc(O)cc1)C(=O)O]

or a prodrug, ester, or pharmaceutically acceptable salt or solvate thereof.

In another embodiment of any of the above uses or methods, the bacteria may comprise S. aureus, and the compound may comprise formula 1

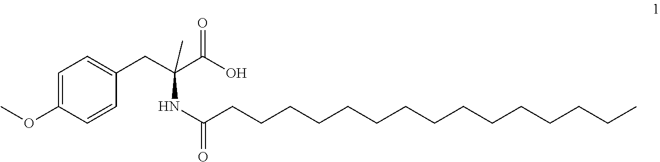 [SMILES: CCCCCCCCCCCCCCCC(=O)N[C@](C)(Cc1ccc(OC)cc1)C(=O)O]

or a prodrug, ester, or pharmaceutically acceptable salt or solvate thereof;
formula 3

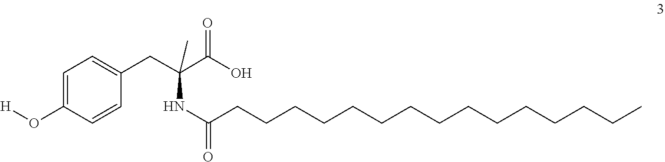 [SMILES: CCCCCCCCCCCCCCCC(=O)N[C@](C)(Cc1ccc(O)cc1)C(=O)O]

or a prodrug, ester, or pharmaceutically acceptable salt or solvate thereof;
or both.

In another embodiment of any of the above uses or methods, the bacteria may comprise an Enterococcus or Staphylococcus and the compound may comprise an α-methyl substituent (i.e. R₁═—CH₃).

In another embodiment of any of the above uses or methods, the bacteria may comprise an Enterococcus and the compound may comprise an O-methylation at the tyrosine side chain (i.e. R₂═—CH₃).

In another embodiment of any of the above uses or methods, the bacteria may comprise a Staphylococcus and the compound may comprise a tyrosine side chain (i.e. R₂═—H).

In another embodiment, there is provided herein a bacterial sample comprising Alteromonas RKMC-009. In yet another embodiment, there is provided herein a sample comprising Alteromonas RKMC-009 deposited under NRRL accession number NRRL B-67979, or a functional equivalent thereof.

In another embodiment, there is provided herein a method for producing a compound as described herein, said method comprising:
providing a compound of formula C:

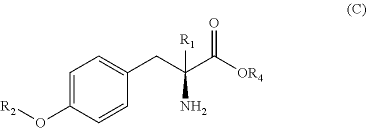

wherein
R₁ is —H; or a linear or branched, optionally substituted, C₁-C₆ alkyl; or optionally substituted phenyl;
R₂ is —H; or a linear or branched, optionally substituted, C₁-C₆ alkyl; or optionally substituted phenyl;
R₄ is —H; linear or branched, optionally substituted, C₁-C₆ alkyl; or optionally substituted phenyl; and
performing an N-acylation with a linear or branched, optionally substituted, C₆-C₂₁ acyl chloride.

In another embodiment of the above method, the compound may comprise formula 1

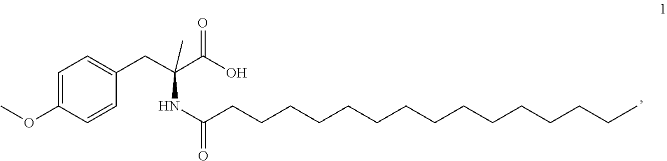 [SMILES: CCCCCCCCCCCCCCCC(=O)N[C@](C)(Cc1ccc(OC)cc1)C(=O)O]

and the method may comprise reacting O-methyl-α-methyl-L-tyrosine with palmitoyl chloride for N-acylation.

In another embodiment of the above method, the compound may comprise formula 2

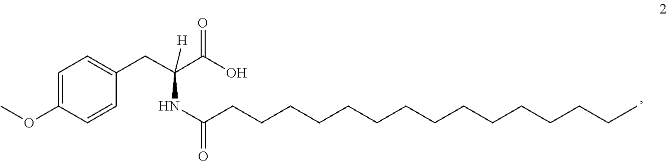 [SMILES: CCCCCCCCCCCCCCCC(=O)N[C@H](Cc1ccc(OC)cc1)C(=O)O]

and the method may comprise reacting O-methyl-L-tyrosine with palmitoyl chloride for N-acylation.

In another embodiment of the above method, the compound may comprise formula 3

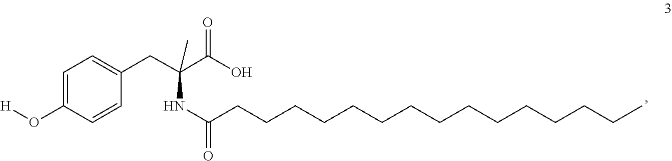 [SMILES: CCCCCCCCCCCCCCCC(=O)N[C@](C)(Cc1ccc(O)cc1)C(=O)O]

and the method may comprise reacting α-methyl-L-tyrosine with palmitoyl chloride for N-acylation.

In another embodiment of the above method, the compound may comprise formula 4

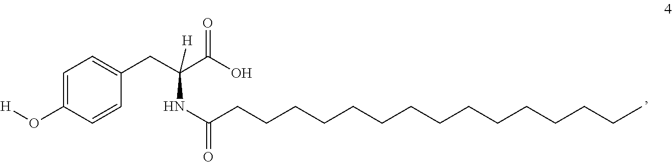 [SMILES: CCCCCCCCCCCCCCCC(=O)N[C@H](Cc1ccc(O)cc1)C(=O)O]

and the method may comprise reacting L-tyrosine with palmitoyl chloride for N-acylation.

In another embodiment, there is provided herein a method for producing a compound of formula 1, said method comprising:
fermenting Alteromonas sp. RKMC-009 bacteria in BFM4m broth; and
extracting the broth with EtOAc.

In another embodiment of the above method, the method may further comprise purifying the compound of formula 1 by reverse phase chromatography.

In another embodiment of any of the above method or methods, the Alteromonas sp. RKMC-009 bacteria is Alteromonas RKMC-009 deposited under NRRL accession number NRRL B-67979, or a functional equivalent thereof.

In another embodiment, there is provided herein a lysate, supernatant, broth, or extract derived or prepared from an Alteromonas sp. RKMC-009 bacterial culture or ferment, wherein the lysate, supernatant, broth, or extract comprises a compound of formula 1:

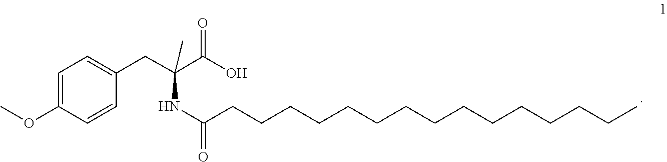 [SMILES: CCCCCCCCCCCCCCCC(=O)N[C@](C)(Cc1ccc(OC)cc1)C(=O)O]

In another embodiment of the lysate, supernatant, broth, or extract, the Alteromonas sp. RKMC-009 bacterial culture or ferment may comprise Alteromonas sp. RKMC-009 deposited under NRRL accession number NRRL B-67979, or a functional equivalent thereof.

### BRIEF DESCRIPTION OF DRAWINGS

FIG. 1 shows (A) A diagram of the ichip fabricated for this study. (B) Xestospongia muta with ichips implanted in its tissue. Five individual ichips are visible in this portion of the sponge and each is marked with a white arrow. Colored tags adjacent to each ichip were used as identifiers (Scale bars: 2.0 cm);

FIG. 2 shows N-Palmitoyl-α,O-dimethyl-L-tyrosine (Compound (1)) with all COSY correlations and key HMBC NMR correlations. Additional correlations are presented in Table 1;

FIG. 3 shows a proposed biosynthetic pathway for the production of compound (1) (ACP=acyl carrier protein, NAS=N-acyl amino acid synthase, MT=methyltransferase);

FIG. 4 shows ultra high pressure liquid chromatography-high resolution mass spectrometry (UHPLC-HMRS) analysis of the ethyl acetate extract of a 5 mL culture of Alteromonas sp. RKMC-009. Compound 1 elutes at 5.37 min (PDA=photodiode array, ELSD=evaporative light-scattering detector, TIC=total ion current);

FIG. 5 shows neighbour-joining tree based on 38 16S rRNA gene sequence, which shows the phylogenetic relationships among Alteromonas sp. RKMC-009 and selected taxa from the Alteromonadaceae. Numbers at the nodes indicate bootstrap values (1000 replicates) for maximum parsimony, unweighted pair group method and arithmetic mean, maximum likelihood and neighbour-joining analyses (respectively). Only nodes with bootstrap values exceeding 50% in at least one analysis are shown. A dash indicates the node was not observed. Pseudoalteromonas haloplanktis was used as the outgroup. Scale bar=0.01 changes per nucleotide position;

FIG. 6 shows small-scale fermentations of Alteromonas sp. RKMC-009 demonstrating the requirement of marine salts for this strain to grow. Growth media with (ISP2) and without marine salts [ISP2m; 18 g/L Instant Ocean (Spectrum Brands)] were inoculated with multiple colonies of RKMC-009. Growth was observed only in ISP2m. Blank cultures were not inoculated and are included for comparison to those that were inoculated with RKMC-009;

FIG. 7 shows ¹H NMR spectrum (400 MHz, CDCl₃) of compound 1;

FIG. 8 shows DEPTQ-135 NMR spectrum (101 MHz, CDCl₃) of compound 1;

FIG. 9 shows COSY NMR spectrum (400 MHz, CDCl₃) of compound 1;

FIG. 10 shows HSQC NMR spectrum (¹H 400 MHz, ¹³C 101 MHz, CDCl₃) of compound 1. Blue contours are positively phased (methyl and methine) and red contours are negatively phased (methylene);

FIG. 11 shows HMBC NMR spectrum (¹H 400 MHz, ¹³C 101 MHz, CDCl₃) of compound 1;

FIG. 12 shows NOESY NMR spectrum (400 MHz, CDCl₃) of compound 1;

FIG. 13 shows Marfey's analysis of compound 1. Extracted ion chromatograms (m/z 448.1463) of L-FDAA-derivatized (A) 1 (hydrolysate), (B) α,O-dimethyl-DL-tyrosine (hydrolysate) and (C) α-methyl-L-tyrosine with the corresponding mass spectra in (D)-(F) (respectively);

FIG. 14 shows ¹H NMR spectrum of compound 1 (synthetic);

FIG. 15 shows DEPTQ-135 NMR spectrum of compound 1 (synthetic);

FIG. 16 shows ¹H NMR spectrum of compound 2;

FIG. 17 shows DEPTQ-135 NMR spectrum of compound 2;

FIG. 18 shows ¹H NMR spectrum of compound 3;

FIG. 19 shows DEPTQ-135 NMR spectrum (101 MHz, CDCl₃) of compound 3;

FIG. 20 shows ¹H NMR spectrum (400 MHz, DMSO-d₆) of compound 4;

FIG. 21 shows DEPTQ-135 NMR spectrum of compound 4;

FIG. 22 shows ¹H NMR spectrum of compound 5;

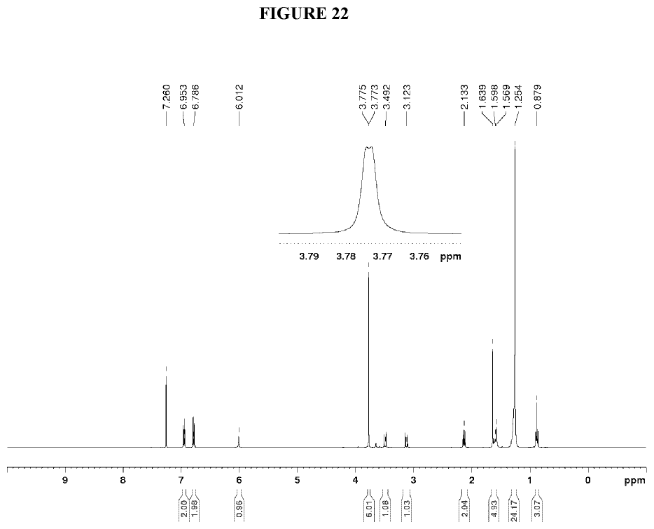

FIG. 23 shows DEPTQ-135 NMR spectrum (101 MHz, CDCl₃) of compound 5;

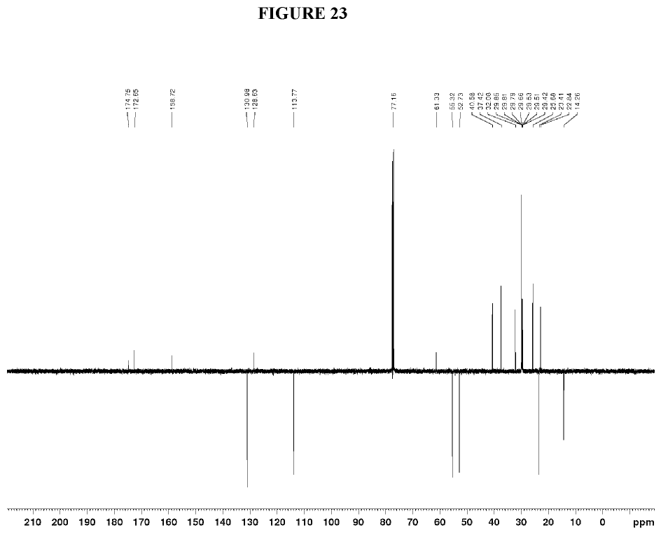

FIG. 24 shows antimicrobial activity of compounds 1-4 against six Enterococcus spp. Enterococcus inhibitory activity of compounds 1-4 expressed as 95% confidence intervals around mean IC₅₀ values (N=3). Data are not included for the following: E. faecium 15337 (4; inactive), E. faecalis 16371 (2; inactive) and E. faecalis 16371 (3; estimated IC₅₀=9.2-18.4 μM);

FIG. 25 shows dose-response curves for compounds 1 and 3 against two Staphylococcus spp.; and

FIG. 26 shows identification of compounds 3 and 4 in the culture extract of Alteromonas sp. RKMC-009 using UHPLC HRMS.

### DETAILED DESCRIPTION

Described herein are N-acyl-tyrosine derivatives, and uses thereof as antibacterial agents. It will be appreciated that embodiments and examples are provided for illustrative purposes intended for those skilled in the art, and are not meant to be limiting in any way.

In an embodiment, there is provided herein a compound of formula A:

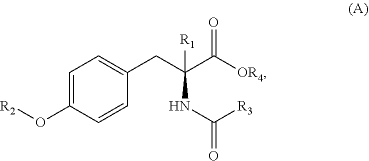

wherein
   R₁ is —H; or a linear or branched, optionally substituted, C₁-C₆ alkyl; or optionally substituted phenyl;
   R₂ is —H; or a linear or branched, optionally substituted, C₁-C₆ alkyl; or optionally substituted phenyl;
   R₃ is linear or branched, optionally substituted, C₅-C₂₀ alkyl, alkenyl, or alkynyl; and
   R₄ is —H; linear or branched, optionally substituted, C₁-C₆ alkyl; or optionally substituted phenyl;
or a prodrug, ester, or pharmaceutically acceptable salt or solvate thereof.

In certain embodiments, the compound may be a compound of formula 1:

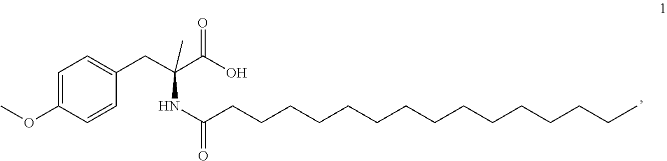 [SMILES: CCCCCCCCCCCCCCCC(=O)N[C@](C)(Cc1ccc(OC)cc1)C(=O)O]

or a prodrug, ester, or pharmaceutically acceptable salt or solvate thereof. In further embodiments, the compound is not a compound of formula 1.

As will be understood, a linear or branched, optionally substituted, C₁-C₆ alkyl group at R₁ or R₂ may comprise any suitable linear or branched C₁-C₆ alkyl group which may, optionally, be further substituted with one or more substituents such as, but not limited to, halogen (i.e. F, Cl, Br, I), hydroxyl, —C≡N, C₁-C₆ alkyl, C₂-C₆ alkenyl, or C₂-C₆ alkynyl, for example. As will also be understood, an optionally substituted phenyl group at R₁ or R₂ may comprise a phenyl group which may, optionally, be further substituted with one or more substituents such as, but not limited to, halogen (i.e. F, Cl, Br, I), hydroxyl, —CN, C₁-C₆ alkyl, C₂-C₆ alkenyl, or C₂-C₆ alkynyl, for example. In certain embodiments, R₁ and R₂ may be each independently selected from —H, Me, Et, nPr, iPr, tBu, iBu, secBu, nBu, or Ph.

In certain embodiments, linear or branched, optionally substituted, C₅-C₂₀ alkyl, alkenyl, or alkynyl at R₃ may comprise any suitable linear or branched C₅-C₂₀ alkyl, alkenyl, or alkynyl (i.e. C₅-C₂₀ group optionally having 0, 1, 2, or more double and/or triple carbon-carbon bonds) which may, optionally, be further substituted with one or more substituents such as, but not limited to, halogen (i.e. F, Cl, Br, I), hydroxyl, —CN, C₁-C₆ alkyl, C₂-C₆ alkenyl, or C₂-C₆ alkynyl, for example. In certain embodiments, R₃ may be a C₅-C₂₀ group with 0-3 double and/or triple carbon-carbon bonds (0-3 Δ). In certain embodiments, R₃ is a linear C₅-C₂₀ group with 0-3 double and/or triple carbon-carbon bonds (0-3 A).

As will also be understood, a linear or branched, optionally substituted, C₁-C₆ alkyl group at R₄ may comprise any suitable linear or branched C₁-C₆ alkyl group which may, optionally, be further substituted with one or more substituents such as, but not limited to, halogen (i.e. F, Cl, Br, I), hydroxyl, —CN, C₁-C₆ alkyl, C₂-C₆ alkenyl, or C₂-C₆ alkynyl, for example. As will also be understood, an optionally substituted phenyl group at R₄ may comprise a phenyl group which may, optionally, be further substituted with one or more substituents such as, but not limited to, halogen (i.e. F, Cl, Br, I), hydroxyl, —C≡N, C₁-C₆ alkyl, C₂-C₆ alkenyl, or C₂-C₆ alkynyl, for example. In certain embodiments, R₄ is —H, Me, Et, or Ph.

As will be understood, the person of skill in the art having regard to the teachings herein will be aware of a variety of prodrugs, esters, or pharmaceutically acceptable salts or solvates of any of the compounds as described herein which may be prepared. In certain embodiments, prodrugs may typically comprise a prodrug moiety which may be linked to the compound through a biocleavable linker. In certain embodiments, esters may typically comprise an alkyl or other moiety which may be linked to the compound through a carboxylic acid or hydroxyl functional group of the compound via an ester linkage. In certain embodiments, pharmaceutically acceptable salts or solvates may include any suitable salts or solvates known to the person of skill in the art having regard to the teachings herein, such as those described in Remington: The Science and Practice of Pharmacy (2006), Remington's Pharmaceutical Sciences (2000—20th edition), and in the United States Pharmacopeia: The National Formulary (USP 24 NF19) published in 1999, each of which are herein incorporated by reference in their entireties.

In certain embodiments, the compound may be a compound of formula B:

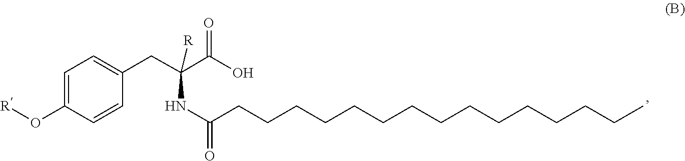

wherein
   R is —CH₃ or —H; and
   R′ is —CH₃ or —H;
or a prodrug, ester, or pharmaceutically acceptable salt or solvate thereof.

In yet another embodiment, the compound may be a compound of formula 1:

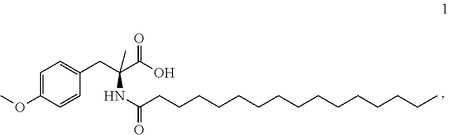 [SMILES: CCCCCCCCCCCCCCCC(=O)N[C@](C)(Cc1ccc(OC)cc1)C(=O)O]

or a prodrug, ester, or pharmaceutically acceptable salt or solvate thereof.

In still another embodiment, the compound may be a compound of formula 2:

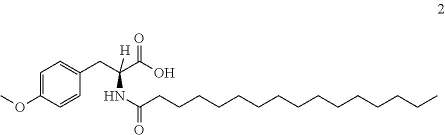 [SMILES: CCCCCCCCCCCCCCCC(=O)N[C@H](Cc1ccc(OC)cc1)C(=O)O]

or a prodrug, ester, or pharmaceutically acceptable salt or solvate thereof.

In yet another embodiment, the compound may be a compound of formula 3:

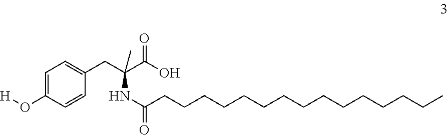 [SMILES: CCCCCCCCCCCCCCCC(=O)N[C@](C)(Cc1ccc(O)cc1)C(=O)O]

or a prodrug, ester, or pharmaceutically acceptable salt or solvate thereof.

In still another embodiment, the compound may be a compound of formula 4:

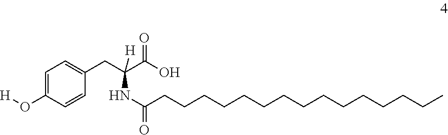 [SMILES: CCCCCCCCCCCCCCCC(=O)N[C@H](Cc1ccc(O)cc1)C(=O)O]

or a prodrug, ester, or pharmaceutically acceptable salt or solvate thereof.

In still another embodiment, there is provided herein a pharmaceutical composition comprising any one or more of the compounds as described herein, and optionally further comprising a pharmaceutically acceptable excipient, diluent, or carrier. Examples of such pharmaceutically acceptable excipients, diluents, and carriers may be found in Remington: The Science and Practice of Pharmacy (2006)). As well, examples of pharmaceutically acceptable carriers, diluents, and excipients may be found in, for example, Remington's Pharmaceutical Sciences (2000—20th edition) and in the United States Pharmacopeia: The National Formulary (USP 24 NF19) published in 1999, each of which are herein incorporated by reference in their entireties. In certain embodiments, a pharmaceutically acceptable carrier, diluent, or excipient may include any suitable carrier, diluent, or excipient known to the person of skill in the art. Examples of pharmaceutically acceptable excipients may include, but are not limited to, cellulose derivatives, sucrose, and starch. The person of skill in the art will recognize that pharmaceutically acceptable excipients may include suitable fillers, binders, lubricants, buffers, glidants, and disentegrants known in the art (see, for example, Remington: The Science and Practice of Pharmacy (2006)). Examples of pharmaceutically acceptable carriers, diluents, and excipients may be found in, for example, Remington's Pharmaceutical Sciences (2000—20th edition) and in the United States Pharmacopeia: The National Formulary (USP 24 NF19) published in 1999.

In another embodiment, there is provided herein a use of any of the compound or compounds, or the pharmaceutical composition or compositions, as described herein as an antibacterial agent, or for treating or preventing a bacterial infection of a cell or subject in need thereof. In certain embodiments, the antibacterial agent may be for in vitro or in vivo applications, and/or may be for use as a preservative or disinfectant.

In another embodiment, there is provided herein a method for reducing or preventing growth of a bacteria, said method comprising:
contacting the bacteria with a compound or pharmaceutical composition as described herein.

In another embodiment, there is provided herein a method for treating or preventing a bacterial infection in a subject in need thereof, said method comprising:
administering a compound or pharmaceutical composition as described herein to the subject.

In another embodiment, the bacteria may comprise a Staphylococcus or Enterococcus bacteria. In certain embodiments, the bacteria may comprise Staphylococcus aureus, Staphylococcus warneri, or Enterococcus faecium. In certain embodiments, the bacteria may comprise methicillin-resistant Staphylococcus aureus (MRSA), or vancomycin-resistant Enterococcus faecium (VRE). In another embodiment, the bacteria may comprise E. faecium EF379 (VRE), E. faecium 15337, E. faecalis 16371, E. gallinarum 20993, E. casseliflavus 15984, E. hirae 17446, S. aureus ATCC 33591 (MRSA), or S. warneri ATCC 17917.

In another embodiment, of a use or method as described herein, the bacteria may comprise an Enterococcus and the compound may comprise formula 1

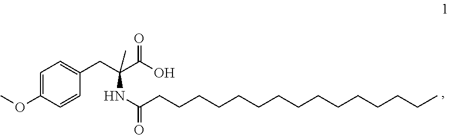 [SMILES: CCCCCCCCCCCCCCCC(=O)N[C@](C)(Cc1ccc(OC)cc1)C(=O)O]

or a prodrug, ester, or pharmaceutically acceptable salt or solvate thereof.

In still another embodiment of a use or method as described herein the bacteria may comprise an Enterococcus and the compound may comprise formula 3

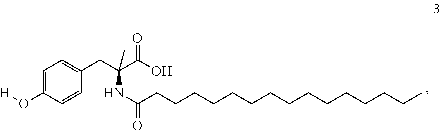 [SMILES: CCCCCCCCCCCCCCCC(=O)N[C@](C)(Cc1ccc(O)cc1)C(=O)O]

or a prodrug, ester, or pharmaceutically acceptable salt or solvate thereof.

In still another embodiment of a use or method as described herein, the bacteria may comprise S. aureus, and the compound may comprise formula 1

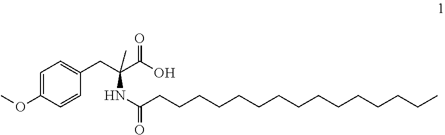 [SMILES: CCCCCCCCCCCCCCCC(=O)N[C@](C)(Cc1ccc(OC)cc1)C(=O)O]

or a prodrug, ester, or pharmaceutically acceptable salt or solvate thereof;
formula 3

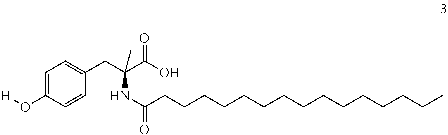 [SMILES: CCCCCCCCCCCCCCCC(=O)N[C@](C)(Cc1ccc(O)cc1)C(=O)O]

or a prodrug, ester, or pharmaceutically acceptable salt or solvate thereof;
or both.

In another embodiment of a use or method as described herein, the bacteria may comprise an Enterococcus or Staphylococcus and the compound may comprise an α-methyl sub stituent (i.e. R₁═—CH₃). In another embodiment of a use or method as described herein, the bacteria may comprise an Enterococcus and the compound may comprise an O-methylation at the tyrosine side chain (i.e. R₂═—CH₃). In still another embodiment of a use or method as described herein, the bacteria may comprise a Staphylococcus and the compound may comprise a tyrosine side chain (i.e. R₂═—H).

In still another embodiment, there is provided herein a bacterial sample comprising Alteromonas RKMC-009. Genome sequence of Alteromonas RKMC-009 may be found in GenBank (CP031010 & CP032914), and is further described in Maclntyre, L. W, Haltli, B. A., Kerr, R. G., Microbiology Resource Announcements (2019), 8(25): 2445, which is herein incorporated by reference in its entirety. Alteromonas sp. RKMC-009 has been deposited with the Agricultural Research Culture Collection (NRRL) International Depositary Authority on Aug. 17, 2020 (the assigned “date of the original deposit”), and assigned Accession Number NRRL B-67979. Copies of the “Receipt in the Case of an Original Deposit” and the “Viability Statement” issued by the IDA, as well as FORM PCT/RO/134, are included below. In an embodiment, there is provided herein a sample comprising Alteromonas RKMC-009 deposited under NRRL accession number NRRL B-67979, or a functional equivalent thereof.

In another embodiment, there is provided herein a method for producing a compound as described herein, said method comprising:
providing a compound of formula C:

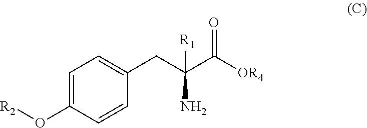

wherein
   R₁ is —H; or a linear or branched, optionally substituted, C₁-C₆ alkyl; or optionally substituted phenyl;
   R₂ is —H; or a linear or branched, optionally substituted, C₁-C₆ alkyl; or optionally substituted phenyl;
   R₄ is —H; linear or branched, optionally substituted, C₁-C₆ alkyl; or optionally substituted phenyl; and
performing an N-acylation with a linear or branched, optionally substituted, C₆-C₂₁ acyl chloride.

In another embodiment, the N-acylation may be performed with any other suitable reagent under conditions suitable to provide for N-acylation with a desired group. For example, N-acylation may be easily performed by reacting the appropriate acyl chloride with the desired amino acid. If the acyl chloride in question is not commercially available, it may be prepared by reaction of the corresponding carboxylic acid with thionyl chloride. Alternatively, in certain embodiments, the corresponding carboxylic acid may be directly coupled to the amino acid using, for example, DCC (dicyclohexylcarbodiimide). Although installation of protecting groups was not required for the preparation of compounds 1-4 as described herein, protecting group(s) may be used in certain embodiments, particularly where preparation of congeners containing amines within R₁, R₂, and/or R₄ is desired, for example.

In certain embodiments, the method may be used to prepare a compound of formula 1

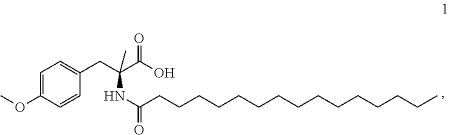 [SMILES: CCCCCCCCCCCCCCCC(=O)N[C@](C)(Cc1ccc(OC)cc1)C(=O)O]

and the method may optionally comprise reacting O-methyl-α-methyl-L-tyrosine with palmitoyl chloride for N-acylation.

In another embodiment, the method may be used to prepare a compound of formula 2

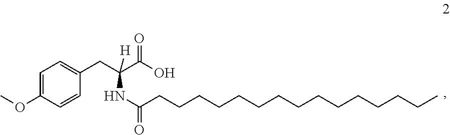 [SMILES: CCCCCCCCCCCCCCCC(=O)N[C@H](Cc1ccc(OC)cc1)C(=O)O]

and the method may optionally comprise reacting O-methyl-L-tyrosine with palmitoyl chloride for N-acylation.

In still another embodiment, the method may be for preparing a compound of formula 3

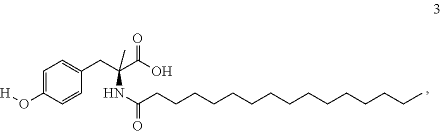 [SMILES: CCCCCCCCCCCCCCCC(=O)N[C@](C)(Cc1ccc(O)cc1)C(=O)O]

and the method may optionally comprise reacting α-methyl-L-tyrosine with palmitoyl chloride for N-acylation.

In yet another embodiment, the method may be for preparing a compound of formula 4

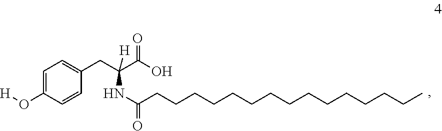 [SMILES: CCCCCCCCCCCCCCCC(=O)N[C@H](Cc1ccc(O)cc1)C(=O)O]

and the method may optionally comprise reacting L-tyrosine with palmitoyl chloride for N-acylation.

In still another embodiment, there is provided herein a method for producing a compound of formula 1, said method comprising:
fermenting Alteromonas sp. RKMC-009 bacteria in BFM4m broth; and
extracting the broth.

In certain embodiments, the broth may be extracted with EtOAc. In another embodiment, the method may further comprise a step of purifying the compound of formula 1 by reverse phase chromatography.

In another embodiment of any of the above method or methods, the Alteromonas sp. RKMC-009 bacteria may be or comprise Alteromonas RKMC-009 deposited under NRRL accession number NRRL B-67979, or a functional equivalent thereof.

In still another embodiment, there is provided herein a lysate, supernatant, broth, or extract derived or prepared from an Alteromonas sp. RKMC-009 bacterial culture or ferment, wherein the lysate, supernatant, broth, or extract comprises a compound of formula 1:

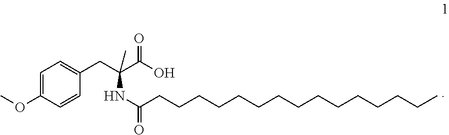 [SMILES: CCCCCCCCCCCCCCCC(=O)N[C@](C)(Cc1ccc(OC)cc1)C(=O)O]

In another embodiment of the lysate, supernatant, broth, or extract, the Alteromonas sp. RKMC-009 bacterial culture or ferment may comprise Alteromonas sp. RKMC-009 deposited under NRRL accession number NRRL B-67979, or a functional equivalent thereof.

For further clarity, it will be understood that compounds of formula A (such as the compound of formula 1) and formula C as set out herein may be equivalently drawn as follows using conventional wedge-and-dash notation:

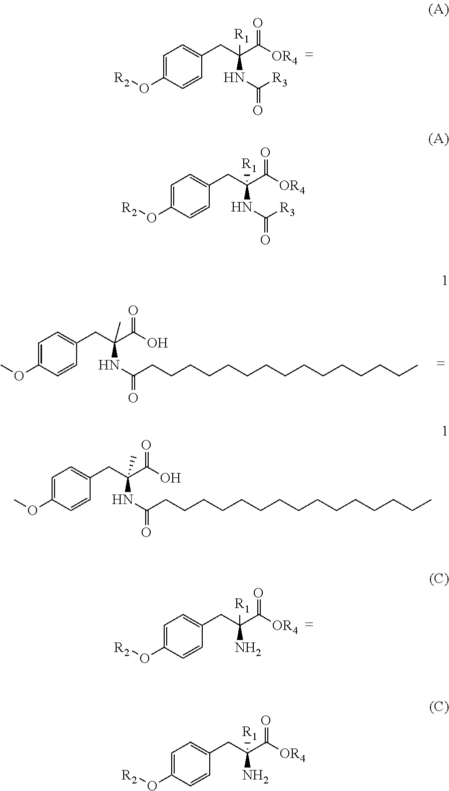

### EXAMPLE 1

### iChip Domestication of Sponge Bacterium Producing a Functionalized N-Acyl-Tyrosine Derivative (N-Acyltyrosine Bearing an α-Methyl Substituent), and Studies Thereof

The vast majority of environmental bacteria that have yet to be cultured in the laboratory may produce an attractive pool of natural products for antimicrobial discovery. In situ cultivation was recently popularized for cultivating uncultured bacteria through development of the ichip (isolation chip). The ichip may allow for high-throughput in situ bacterial cultivation (and simultaneous isolation) and in the nearly decade since its description, there have been remarkably few reports of its use.

The present study employed the isolation chip (ichip) in what is believed to be for the first time in a marine sponge (Xestospongia muta) that harbours a rich microbiome, and describes the subsequent isolation of what is believed to be a new bacterial species, Alteromonas sp. RKMC-009. As described in detail herein, it has been discovered that strain RKMC-009 produces a structurally unique N-acyl amino acid, which is an N-acyltyrosine compound of formula (1) that is functionalized/appended with a rare α-methyl substituent within the amino acid residue (aminoacyl moiety), and also exhibits Gram-positive antibacterial activity. Through an SAR experiment, it is determined that the α-methyl may be a key contributor to potent antibacterial activity, may provide for Staphylococcus activity of compound (1), and may enhance Enterococcus activity.

Despite the success of the ichip for cultivation of new microbial taxa, there have been remarkably few reports describing its use in the almost decade since it was first described. Furthermore, reports of its use are seemingly limited to soil or sediment environments. Accordingly, this study implements the ichip within a tropical marine sponge in an effort to both further the scope of its application, and to uncover new bacterial taxa for natural product discovery. The uncultured microbial diversity of marine sponges has been extensively described and cultured bacteria from these habitats may produce potent bioactive natural products. (6, 7) The high microbial abundance sponge Xestospongia muta was chosen as a subject for this study, primarily for its size; it was expected that it may harbour many ichips with minimal tissue damage. Additionally, X. muta is known to harbour a rich bacterial community. (6, 7)

Ichips were assembled with inoculum prepared from X. muta samples and allowed to incubate within the sponge tissue. The ichips were disassembled in the laboratory and as many bacteria from them were domesticated as possible, after which selected isolates were fermented in culture broth and their extracts screened chemically for the production of potentially new natural products. One particular bacterial isolate, an apparently new species of the genus Alteromonas produced a N-acylated amino acid (1) that exhibited growth inhibitory activity against Gram-positive bacterial pathogens as described in detail hereinbelow. Compound (1) possesses a methyl substituent a-position of the amino acid residue, which may be a unique structural feature of this natural product family. The present study describes in detail the ichip domestication of Alteromonas sp. RKMC-009, the absolute chemical structure and chemical synthesis of compound (1), and a structure-activity relationship (SAR) in which the α-methyl substituent of compound (1) may confer or contribute to its Enterococcus and Staphylococcus activity.

Results and Discussion:

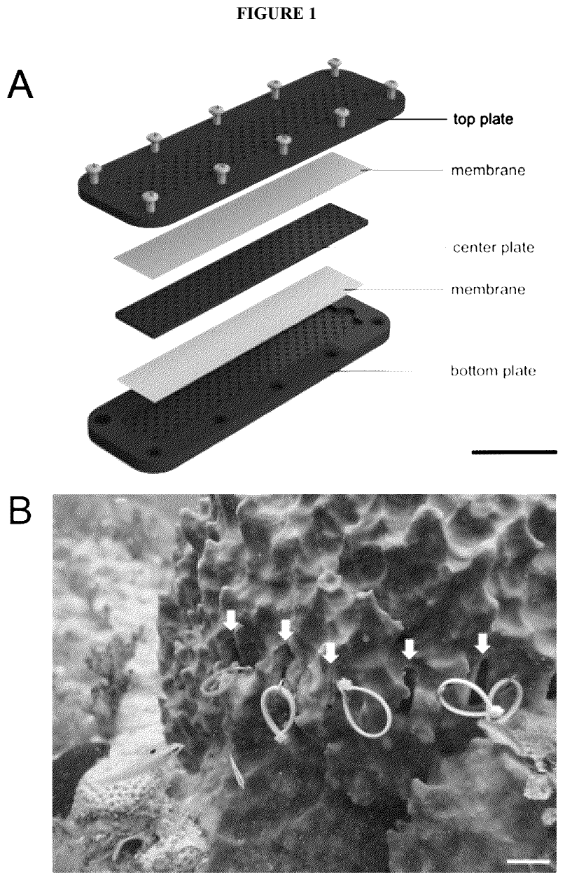

Ichip-domestication of a new Alteromonas sp. Given that they were not commercially available (perhaps a contributing factor the lack of reports describing their use), ichips were fabricated in-house following the existing design as closely as possible with two notable exceptions. First, the thickness of our assembled ichips was reduced to 5.0 mm compared to the original design (14.0 mm) in order to minimize potential damage to X. muta upon implantation of the device in its tissue. Second, the number of through-holes in the center plate of the devices was reduced from 384 to 148 (4). The resulting modified ichips are depicted in FIG. 1A. The seals of randomly sampled ichips were validated to ensure that bacteria could neither enter nor escape the devices when fully assembled. The ichips were implemented in San Salvador, Bahamas in an individual sponge accessed by SCUBA diving on a local reef.

A tissue sample was collected from X. muta and a suspension of its constituent bacteria in diluted complex medium was prepared using a reported protocol. (6) It was important for isolation of pure colonies directly from the ichip that the bacterial suspension be diluted to a defined cell density affording approximately one cell per ichip through-hole. However, the field laboratory lacked a fluorescence microscope or an equally accurate means for enumerating bacterial cells. It was attempted to circumvent this technical challenge by first estimating the total bacterial cell count within the sponge-derived suspension as 8.2×109 cells/mL using reported values of microbial abundance within X. muta. Five dilutions were then prepared from this suspension that spanned the desired cell density (1000 cells/mL) to achieve approximately one cell per through-hole: 5000, 2500, 1000, 500 and 100 cells/mL. Each dilution was used to inoculate duplicate ichips that were then implanted X. muta and incubated for 7 d (FIG. 1B). It was postulated that given the inaccuracy of the enumeration method, perhaps only ichips inoculated with a specific dilution would contain one cell per through-hole.

All ichips were disassembled and processed according to the reported protocol upon their return. (4) Bacterial growth was observed from the through-holes of all ten ichips and a total of 50 morphologically distinct bacterial colonies were isolated; most isolates required purification by serial subculturing suggesting that all ten ichips were inoculated with greater than one cell per through-hole. Although this forbid subsequent isolation of pure cultures, it did not preclude in situ cultivation.

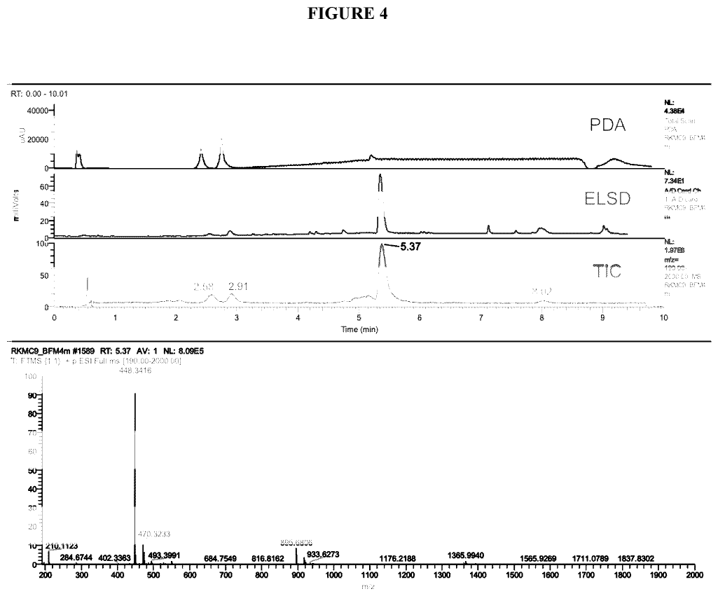

All bacterial isolates were taxonomically identified by sequence analysis (16S rRNA gene) and selected isolates were fermented in seven different growth media in search of potentially new natural products. All culture extracts were analyzed by UHPLC-HRMS and ions of interest were queried in AntiBase 2017. One particular isolate, Alteromonas sp. RKMC-009, was identified as a new species belonging to the genus Alteromonas and was flagged for further analysis due to the presence of a major peak at 5.35 min in the HRMS and ELSD chromatograms (FIG. 4); the peak was associated with a compound (1) with an m/z of 448.3416 [M+H]⁺. A search of Anti-Base 2017 revealed this pseudomolecular ion may be novel. Alteromonas sp. RKMC-009 has been deposited with the Agricultural Research Culture Collection (NRRL) International Depositary Authority on Aug. 17, 2020 (the assigned “date of the original deposit”), and assigned Accession Number NRRL B-67979. Copies of the “Receipt in the Case of an Original Deposit” and the “Viability Statement” issued by the IDA, as well as FORM PCT/RO/134, are included below.

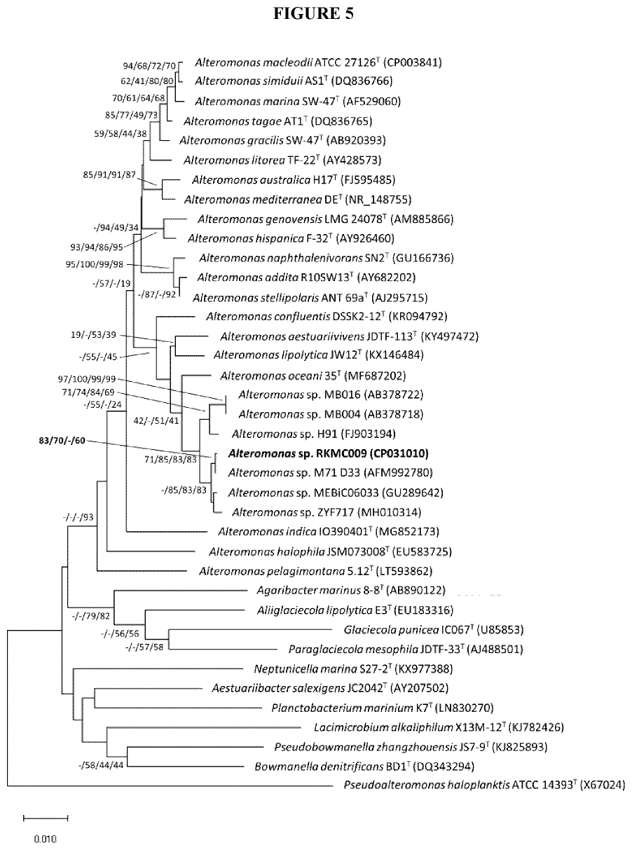

The closest related type strain to RKMC-009 is Alteromonas aesuariivivens JDTF-113 with 98.15% sequence similarity (16S rRNA gene).⁸ A phylogenetic tree was constructed from 16S rRNA gene sequences of RKMC-009, all validly described Alteromonas spp., representative type strains from additional Alteromonadaceae genera and six Alteromonas strains that are not described at the species level but show strong sequence similarity to RKMC-009 (FIG. 5). RKMC-009 formed a well-supported clade with the latter six strains, which collectively may represent two or more new species; however, a detailed taxonomic assessment to fully establish RKMC-009 as a new species was beyond the scope of this analysis.

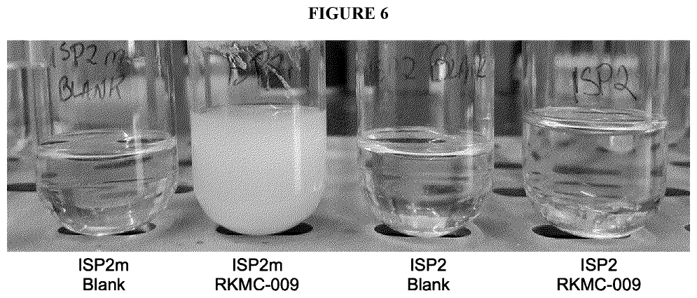

Purification, absolute structure elucidation and chemical synthesis of an α-methylated N-acyltyrosine. Scale-up fermentations of RKMC-009 were carried out in order to purify compound (1), elucidate its chemical structure and assess its biological activities. Alteromonas sp. RKMC-009 was fermented 10×1 L cultures in BFM4m broth that were then combined and extracted with EtOAc; the media were supplemented with 18 g/L Instant Ocean as RKMC-009 required salt to grow (FIG. 6). Compound (1) was purified from the EtOAc extract using flash chromatography to afford 350 mg of material sufficiently pure for spectroscopic characterization and biological assays.

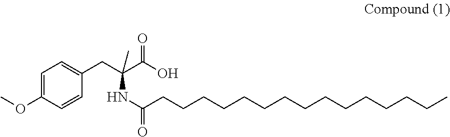 [SMILES: CCCCCCCCCCCCCCCC(=O)N[C@](C)(Cc1ccc(OC)cc1)C(=O)O]

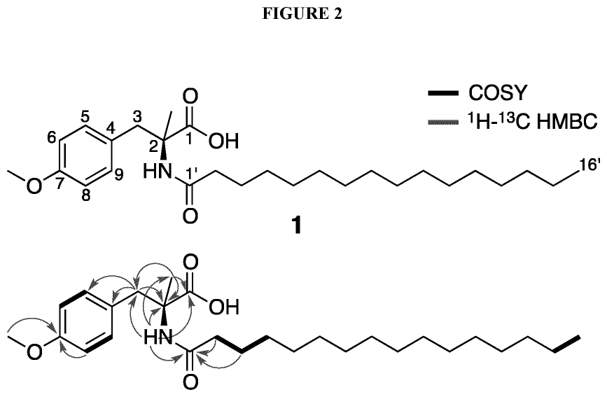

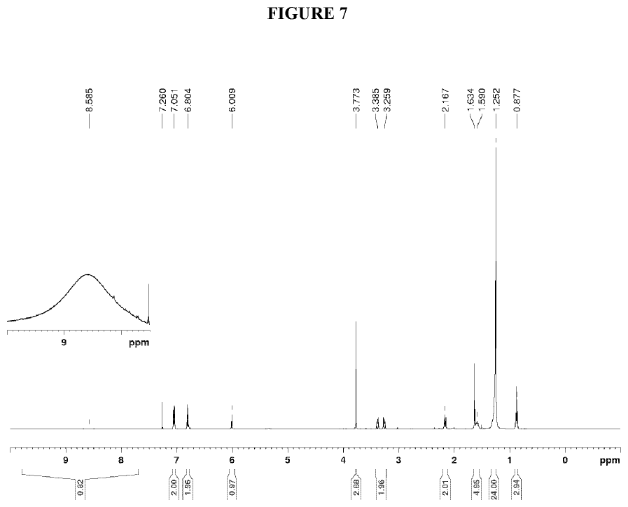

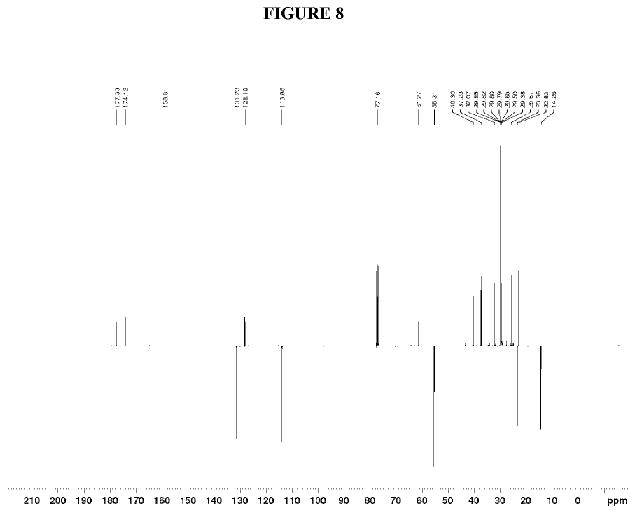

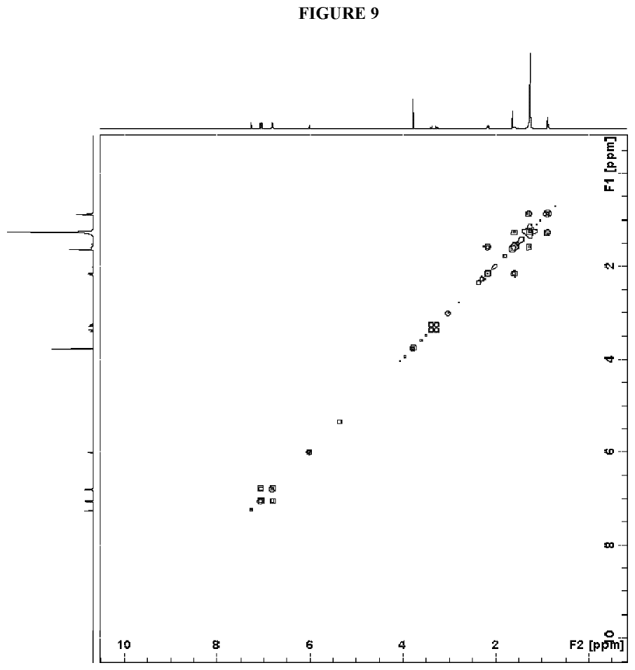

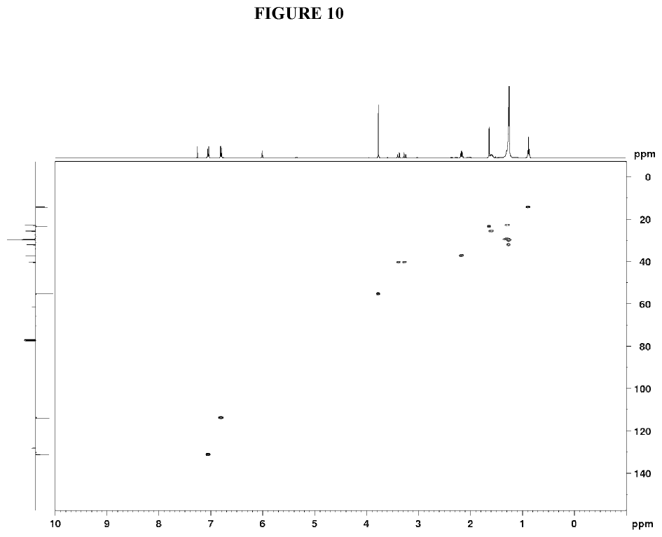

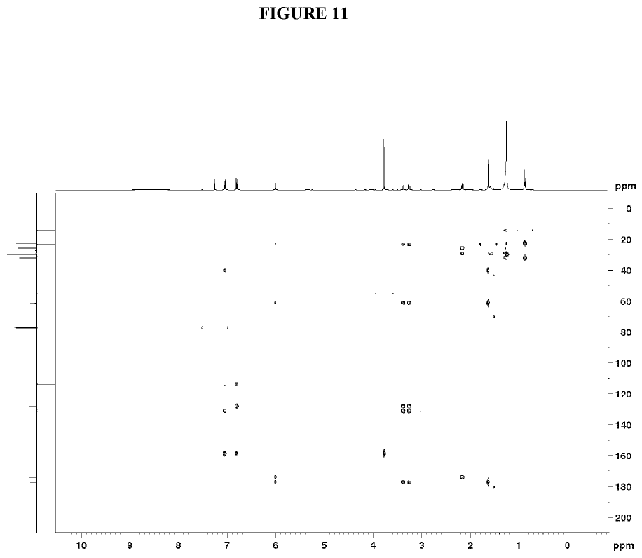

The chemical structure of compound (1) (FIG. 2) was elucidated using a combination of NMR spectroscopic methods and chemical derivatization. Purified compound (1) was obtained as an amorphous white solid and ESI+FIRMS supported a molecular formula of C₂₇H₄₅NO₄, which requires six degrees of unsaturation. The 2D structure of compound (1) was elucidated using a combination of ¹H, DEPTQ-135, COSY, HSQC, and HMBC NMR spectra (FIG. 7-12). Chemical shifts, coupling constants and correlations are presented in Table 2. Initial analyses of the ¹H NMR spectrum immediately suggested the presence of a long aliphatic carbon chain on the basis of a methylene envelope at approximately 1.25 ppm. Correlations in the COSY spectrum between methylenes in this envelope to H-3′ and H-16′ (see FIG. 2), in addition to HMBC NMR correlations from both H-2′ and H-3′ to a ¹³C resonance at 174.1 ppm (C-1′), indicated the presence of a straight chain fatty acyl group. Integration of the methylene envelope allowed to identify this spin system as a palmitoyl (hexadecanoyl) moiety. The remaining molecular formula (C₁₁H₁₂NO₃) after subtraction of a palmitoyl moiety suggested the presence of an amino acid residue containing an oxygenated side chain; moreover, aromatic resonances in the ¹H NMR spectrum (6.80 and 7.05 ppm) strongly indicated a tyrosine-like structure. The presence of a carboxylic acid was supported by a broad singlet at 8.59 ppm in the ¹H NMR spectrum and an amide proton signal was observed at 6.01 ppm. An HMBC NMR correlation from the amide proton to C-1′ indicated that compound (1) is an N-acylated amino acid. Correlations in the HMBC NMR spectrum supported a tyrosine backbone with two notable modifications. First, the side chain hydroxy group is methylated giving rise to a methoxy signal at 3.77 ppm in the ¹H NMR spectrum; an HMBC correlation was observed from this methoxy group to C-7. Second, the singlet amide proton resonance indicated the absence of an α-H. The remaining signal in the NMR spectrum, a singlet at 1.64 ppm integrating to 3-H, allowed for placement of a methyl group at the α-position. This placement was corroborated by HMBC NMR correlations to C-1, C-2 and C-3. HMBC NMR correlations were also observed from the amide proton and both H-3a/H-3b to CH₃-2. Compound (1) satisfies the molecular formula C₂₇H₄₅NO₄ and is consistent with the required degrees of unsaturation.

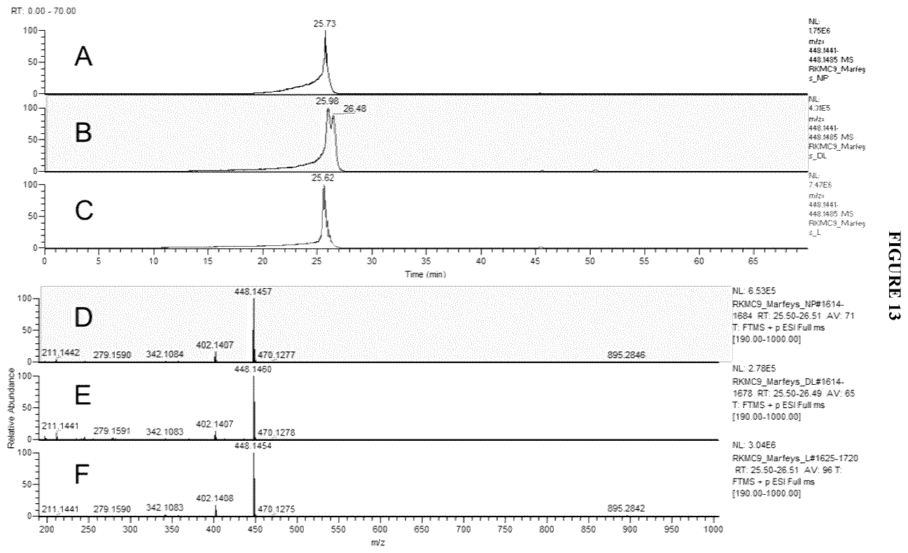

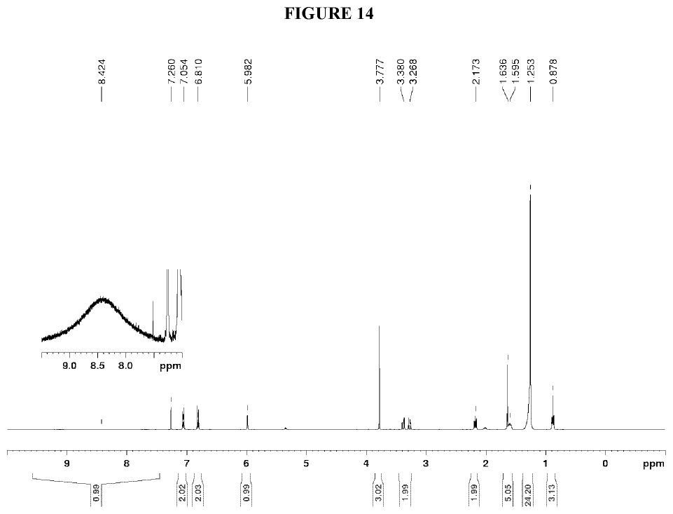

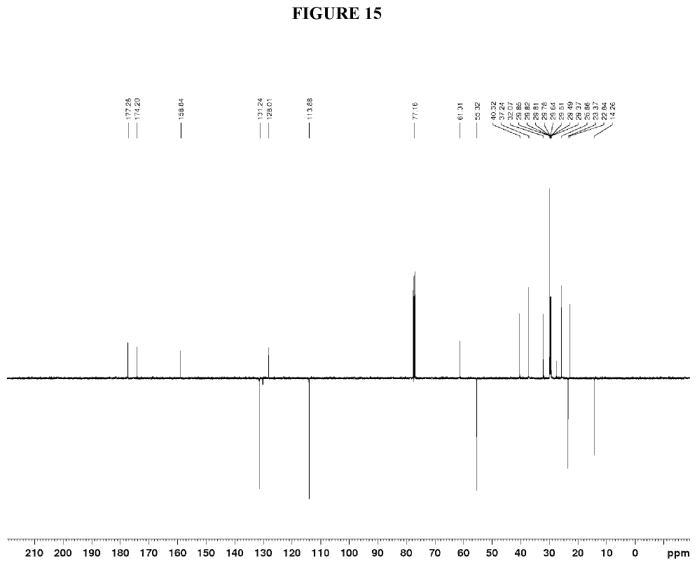

To ensure that OCH₃-7 did not originate from solvolysis of methanol during purification of compound (1) or preparation for UHPLC-HRMS, fermentation of RKMC-009 was repeated on a 5 mL scale in culture tubes (N=3) and analyzed in the absence of methanol. Cultures were extracted with EtOAc and the dried extract was resuspended in CH₃CN for UHPLC-HRMS analysis. Compound (1) was observed in quantities comparable to previous fermentations and it was concluded that Compound (1) is not a solvolysis artifact. Marfey's method was employed to unambiguously assign compound (1) as S-configured at C-2 (N-palmitoyl-α,O-dimethyl-L-tyrosine; FIG. 13). Finally, compound (1) was chemically synthesized from α-methyl-L-tyrosine (Scheme 1) to corroborate the structural assignment. ¹H and ¹³C NMR spectra of synthetic compound (1) (FIG. 14-15) in addition to specific rotation are identical to those of compound (1) purified from bacterial fermentations, thus confirming the absolute structural assignment.

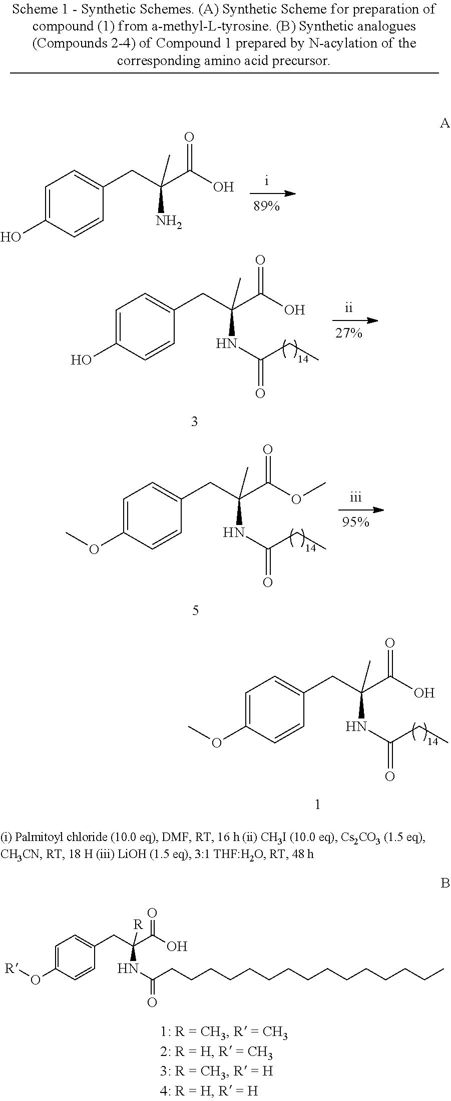

α-Methylation of N-pamitoyltyrosines confers antimicrobial activity. Compound (1) was initially screened for antimicrobial activity against the following panel of pathogenic bacteria and fungi in Table 1 in microbroth dilution assays: methicillin-resistant Staphylococcus aureus (MRSA), Staphylococcus warneri, vancomycin-resistant Enterococcus faecium (VRE), Pseudomonas aeruginosa, Proteus vulgaris and Candida albicans. Growth inhibition was observed against VRE, MRSA and S. warneri while no activity was observed against the Gram-negative or fungal pathogens. The inventors were aware of no reports on anti-Staphylococcus activity of N-acyltyrosines or on anti-Enterococcal activity of any N-acylated amino acid; thus, it was hypothesized that the α-methyl substituent of compound (1) may be a key component of the antimicrobial pharmacophore of compound 1, may confer Staphylococcus inhibitory activity to N-acyltyrosines, and/or perhaps may play a role in their Enterococcus activity.

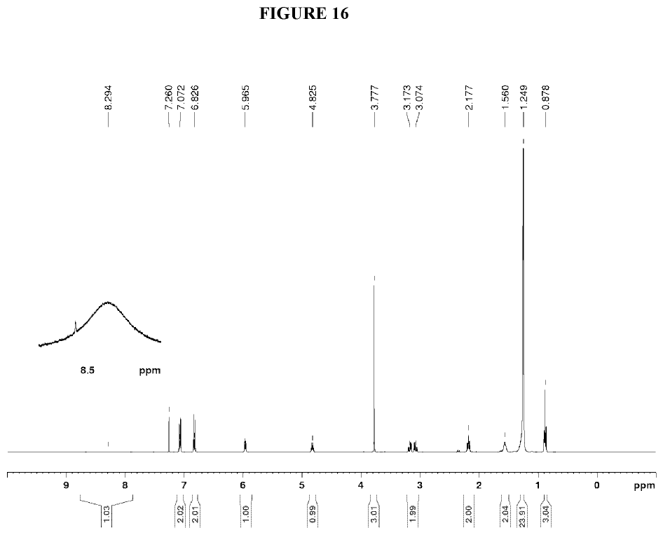

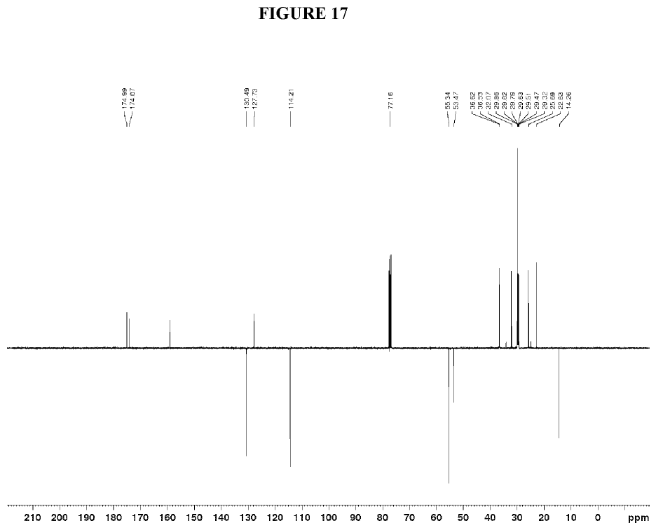

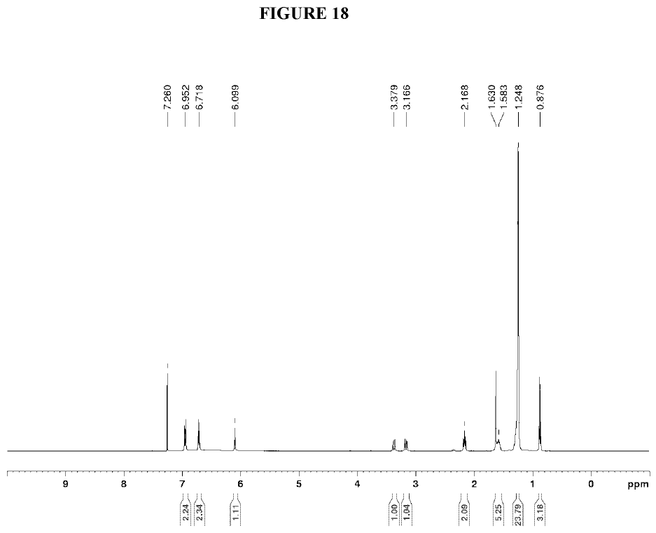

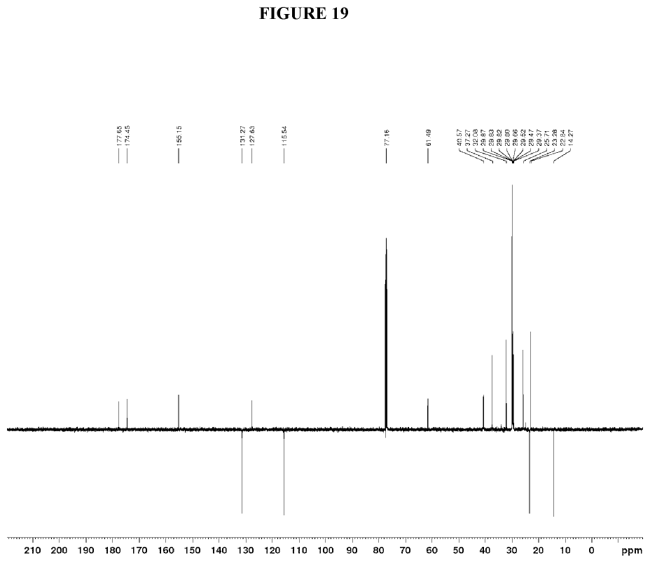

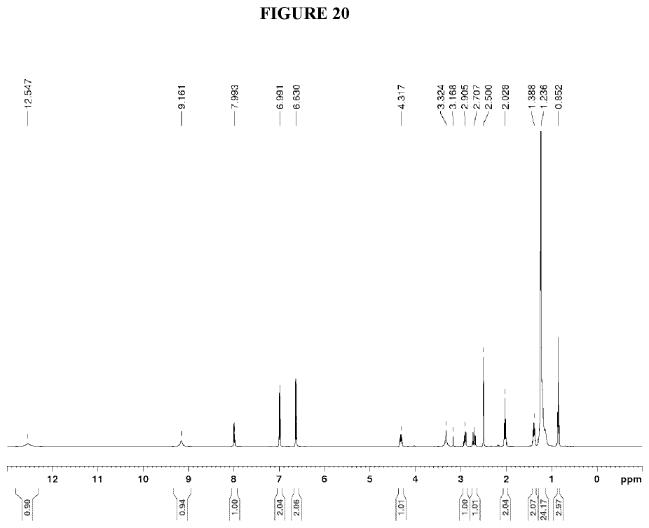

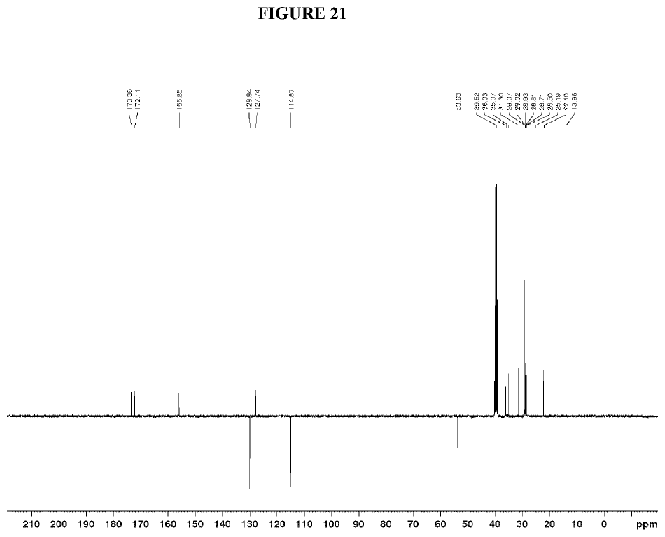

To test the proposed structure-activity relationship (SAR), a structural analogue compound (2) of compound (1) was synthesized that lacks an a-methyl substituent, and compound (2) was tested for antimicrobial activity in parallel with compound (1). Analogues (3) and (4) were also synthesized and assayed to further support a potential SAR involving the O-methyl substituent (FIG. 16-21). To thoroughly characterize the anti-Enterococcal activity of compounds (1)-(4), five veterinary clinical isolates from veterinary cases (in addition to VRE EF379) were obtained for this study. All five clinical isolates were classified as vancomycin-susceptible. (9) The results of this experiment are summarized in Table 1.

TABLE 1
Antibacterial activities of Compound (1) and synthetic analogues (2)-(4).

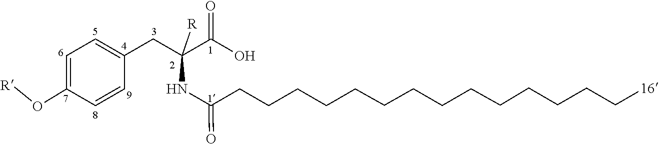

1: R = CH₃, R′ = CH₃
2: R = H, R′ = CH₃
3: R = CH₃, R′ = H
4: R = H, R′ = H

| | IC₅₀^{a}(μM) of N-Acyltyrosines | | | |
|---|---|---|---|---|
| Microbial Pathogen | 1 | 2 | 3 | 4 |
| Enterococcus faecium EF379 (VRE) | 1.6 | 13.3 | 5.3 | 10.7 |
| Enterococcus faecium 15337 | 7.2 | 10.5 | 9.9 | >305.1^{b} |
| Enterococcus faecalis 16371 | 7.4 | >295.2 | 9.2-18.4 | 77.1 |
| Enterococcus gallinarum 20993 | 4.0 | 71.5 | 4.6 | 145.7 |
| Enterococcus casseliflavus 15984 | 3.1 | 10.7 | 9.0 | 32.1 |
| Enterococcus hirae 17446 | 4.1 | 3.6 | 4.6 | 5.6 |
| Staphylococcus aureus ATCC 33591 (MRSA) | >285.9^{b} | >295.2 | 9.2-18.4 | >305.1 |
| Staphylococcus warneri ATCC 17917 | >285.9^{b} | >295.2 | 9.2-18.4 | >305.1 |
| Pseudomonas aeruginosa ATCC 14210 | >285.9 | ND | ND | ND |
| Proteus vulgaris ATCC 12454 | >285.9 | ND | ND | ND |
| Candida albicans ATCC 14035 | >285.9 | ND | ND | ND |

VRE=Vancomycin-resistant Enterococcus, MRSA=methicillin-resistant Staphylococcus aureus, ND=not determined. ^{a}Values are provided as a range when marginal inhibition was observed at the highest concentration tested or data fit the regression model poorly. ^{b}Incomplete growth inhibition was observed from 2-64 μg/mL for S. warneri (<50%), 8-128 μg/mL for S. aureus (<75%) and at all tested concentrations for E. faecium 15337 (<50). Positive controls (antibiotic; IC₅₀): E. faecium EF379 (RIF; 4 μg/mL), E. faecium 15337 (RIF; >16 μg/mL), E. faecalis 16371 (RIF; >8 μg/mL), E. gallinarum 20993 (RIF; 3 μg/mL), E. casseliflavus 15984 (RIF; 0.9 μg/mL), E. hirae 17446 (RIF; <0.5 μg/mL), S. aureus ATCC 33591 (VAN; 2 μg/mL), S. warneri (VAN; <0.125 μg/mL).

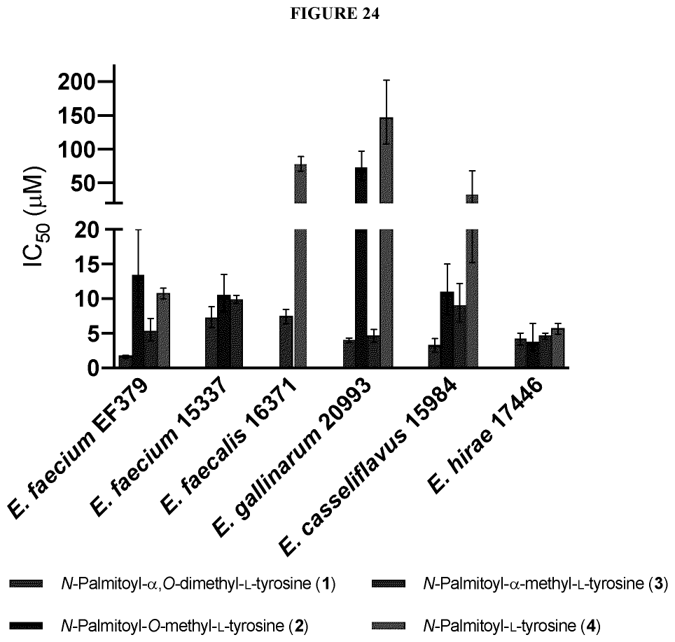

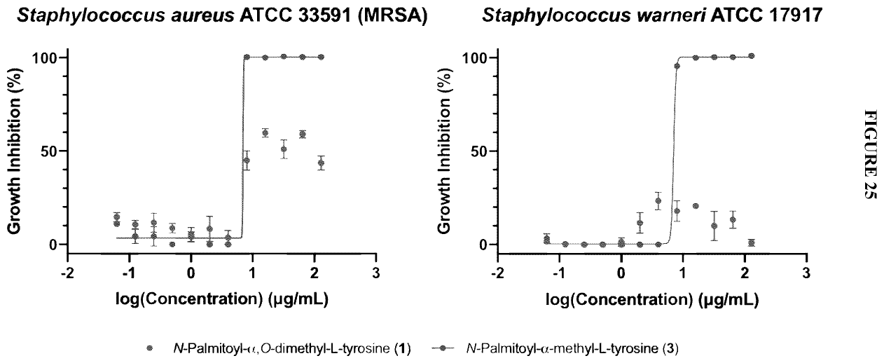

Structure-activity relationships were inferred from pairwise comparison of 95% confidence intervals around mean IC₅₀ values for compounds (1)-(4) (FIG. 24). Compound (1) was more potent (lower IC₅₀) than compound (2) against 4/6 Enterococcus spp. and compound (3) exhibited greater potency than compound (4) against 5/6 Enterococcus spp. Growth of S. aureus and S. warneri were unaffected by compound (2) and compound (4) at the highest concentrations tested (128 μg/mL). S. aureus was inhibited by compound 1 and compound 3, although inhibition by compound 1 was incomplete as maximum inhibition was achieved at 8 ug/mL and plateaued at higher concentrations (FIG. 25). S. warneri was only inhibited by compound 3.

Without wishing to be bound by theory, it was concluded from these observations that the α-methyl may increase inhibitory activity of N-palmitoyltyrosines against most Enterococcus spp. and that it may be important or perhaps even necessary for their Staphylococcus activity. An SAR involving the O-methyl was also observed. Compound (1) was more potent than Compound (3) and compound (2) was more potent than compound (4) against 4/6 and 3/6 Enterococcus spp., respectively. This indicates that O-methylation increased inhibitory activity N-palmitoyltyrosines against Enterococci. Interestingly, the opposite effect was observed against Staphylococcus. Compound (1) was significantly less active than compound (3) against both S. aureus and S. warneri. Compounds (1)-(4) were tested for cytotoxicity against one healthy cell line (normal Cercopithecus aethiops VERO kidney cells) and three human cancer cell lines. Compounds (1)-(4) each exhibited weak inhibitory activity against the cell lines; however, all IC₅₀ values exceeded 50 μM and compounds (1)-(4) were not classified as cytotoxic.

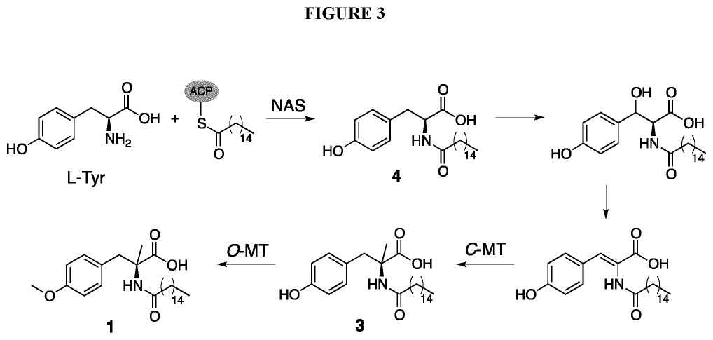

Proposed biosynthetic pathway. Compound (1) is believed to be the first reported N-acyl amino acid bearing an α-alkyl or side chain O-methyl substituent and represents one of only several known structural modifications to the aminoacyl moiety of molecules in this natural product family. Known aminoacyl modifications may include methyl esterification, oxidative decarboxylation to yield enol esters and enamides, and α,β-dehydrogenation. (10-14) Without wishing to be bound by theory, a biosynthetic pathway for the production of (1) is proposed herein (FIG. 3) whereby an α,β-unsaturated intermediate may serve as the substrate for a C-methyltransferase that may install the α-methyl. Congeners of this proposed intermediate, thalassotalic acids A-C, have been reported from a bacterium belonging to the same order (Alteromonadales) as RKMC-009.(14) It may be plausible that an α,β-unsaturated intermediate arises from dehydration of N-palmitoyl-β-hydroxy-L-tyrosine. β-Hydroxytyrosine has been observed within non-ribosomal peptides, as a precursor to non-peptidic metabolites, and support was recently provided for a β-hydroxylation/dehydration sequence in the biosynthesis of a E-2,3-dehydrotyrosine residue found within WS9326A and derivatives.(15-17) It may be that O-methylation was arbitrarily assigned as the last biosynthetic step but may perhaps occur elsewhere in the pathway (including O-methylation of free tyrosine). (18) It should be noted that an alternative pathway may be plausible, in which a radical SAM methyltransferase directly appends N-palmitoyltyrosine with an α-methyl.

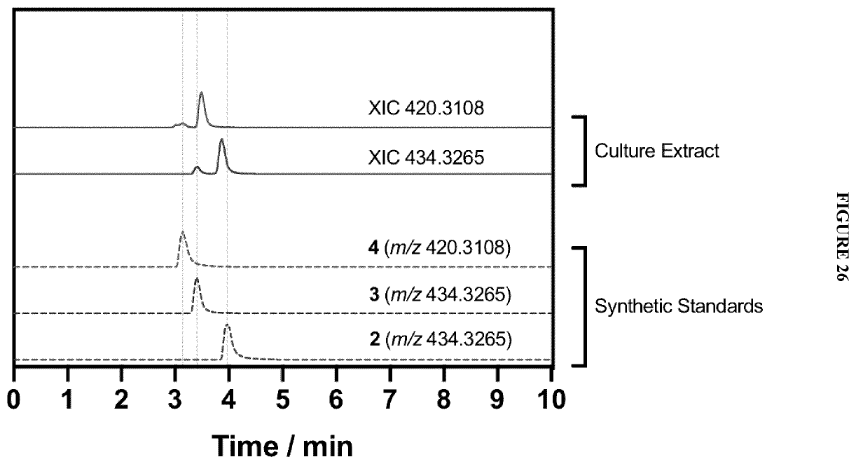

FIG. 26 shows identification of proposed intermediates 3 and 4 in the culture extract of Alteromonas sp. RKMC-009 by comparison (using UHPLC HRMS) to synthetic standards of plausible intermediates 2, 3, and 4 (XIC=Extracted ion chromatogram).

The studies described herein employed a miniaturized ichip to isolate marine bacteria from the tropical marine sponge Xestospongia muta, which may represent perhaps the first application of this device to environments other than soil or sediment. Alteromonas sp. RKMC-009 was domesticated from a particular ichip implanted in X. muta and produces a N-acyltyrosine compound (1) that bears an α-methyl substituent. It was determined through SAR experiments that α-methylation may confer antimicrobial activity to N-palmitoyltyrosines; it was necessary for their Staphylococcus inhibitory activity in the conditions tested and enhanced their Enterococcus activity. A biosynthetic pathway for compound (1) is also proposed herein.

Materials and Methods, Experimental Procedures

General experimental procedures. Optical rotation was measured on a Rudolph Autopol III polarimeter using a 50 mm microcell (1.2 mL). Infrared (IR) spectra were recorded using attenuated total reflectance on a Thermo Nicolet 6700 FT-IR spectrometer. All NMR spectra were acquired on a Bruker Avance III NMR spectrometer (¹H: 400 MHz, ¹³C: 151 MHz) equipped with a 5 mm SmartProbe. All chemical shifts are reported in ppm and referenced to residual solvent signals [¹H (DMSO-d₆): 2.50 ppm, ¹³C (DMSO-d₆): 39.51 ppm, ¹H (CDCl₃): 7.26 ppm, and ¹³C (CDCl₃): 77.16 ppm]. All UHPLC-HRMS analyses (unless otherwise noted) were carried out using the following platform equipped with HRMS-ELSD-UV detection: Thermo Accela UHPLC Pump, Thermo Exactive HRMS fitted with an ESI source, Sedex 80 LT-ELSD, and Thermo PDA. A Kinetex core-shell 100 Å C₁₈ column (2.1×50 mm, 1.7 μm, Phenomenex) was used with a mobile-phase flow rate of 0.5 mL/min and injection volume of 10 μL (all samples were prepared in CH₃OH). The following elution method was used [A=H₂O (0.1% formic acid), B=CH₃CN (0.1% formic acid)]: 5% B from 0.0 to 0.2 min, linear gradient from 5% B at 0.2 min to 99% B at 4.8 min, 99% B from 4.8 to 8.0 min, linear gradient from 99% B at 8.0 min to 5% B at 8.5 min and 5% B from 8.5 to 10.0 min. The following HRMS parameters were used: positive ionization mode, mass resolution of 30,000, mass range of m/z 190 to 2,000, spray voltage of 2.0 kV, capillary temperature of 300° C., S-lens RF voltage of 60.0%, maximum injection time of 10 ms, and 1 microscan. The system was controlled by Thermo Xcalibur software modules. Automated flash chromatography was carried out on a Teledyne ISCO CombiFlash Rf 200 system equipped with UV detection. All reagents were purchased from commercial sources and used without further purification. All solvents used for purification were of HPLC grade or higher.

Ichip fabrication and validation. Twenty ichips were machined from hydrophobic plastic polyoxymethylene and each contains three components: a top and bottom plate (both 75.0×22.0×2.5 mm) and a center plate (63.5×11.5×1.0 mm) with 1.0 mm diameter (1.25 μL) through-holes. Each ichip was fitted with two polycarbonate membranes (Sterlitech; 0.03 μm pore diameter) that were cut with a scalpel to the exact dimensions of the center plate. The following procedure was used for aseptic assembly of the ichips in a laminar flow hood. Membranes were autoclaved and the center and outer plates were sanitized by submersion in isopropanol (70% v/v) for a minimum of 15 min and then the isopropanol was allowed to evaporate in the laminar flow hood before assembling the ichip. All components of the ichips were manipulated with sterilized tweezers. The center plates bearing the growth chamber were inoculated by submerging it in molten (45° C.) agar medium and then gently agitated to ensure all through-holes were filled. The center plates were then removed, the growth medium was allowed to solidify and then the excess agar on the surface of the plates was scraped off using a sterilize glass microscope slide. The ichip components were then assembled as indicated in FIG. 1A and fastened with screws. To validate the ichips' seals and the effectiveness of our aseptic assembly protocol, three randomly chosen ichips were fully assembled such that they contain sterile LB agar with 0.2 mg/mL of 5-bromo-4-chloro-3-indolyl-β-D-galactosidase (X-gal). Each ichip was placed in a 50 mL conical tube containing a 12 h culture (LB broth) of NEB 10-beta cells containing pUC19 (New England BioLabs) and allowed to incubate for 24 h. The ichips contained no visibly blue through-holes after this test indicating external bacteria did not infiltrate the membrane barrier and contaminate the agar plugs in the center plate. Subsequently, three ichips were fully assembled with LB agar inoculated with 3% (v/v) of a 12 h culture (LB broth) of E. coli C3019 containing pUC19 and then placed in a conical tube containing sterile LB broth for 24 h. After the incubation period no bacterial growth was visible in the surrounding culture medium indicating bacteria contained in the ichip are unable to migrate out of the ichip.

Ichip inoculation and incubation. An individual sponge (X. muta) was located at a depth of 10-12 m on Runway 10 Reef (24°03′57.2″N, 74°32′41.3″W) in San Salvador, The Bahamas. Sponge tissue was collected and processed according to a previously reported protocol.¹ A portion of sponge containing outer surface tissue and inner surface tissue was resected using a dive knife and then stored in a sterile plastic bag for transport to our field laboratory for further processing. In the laboratory, a piece of wet sponge tissue weighing approximately 1 g was excised with a sterilized blade, rinsed three times with filter-sterilized (0.2 μm) seawater and then added to 9 mL of filter-sterilized seawater. The tissue was homogenized with a rotor stator that was sterilized by sequentially soaking in 0.525% sodium hypochlorite and isopropanol (70% v/v) for 5 min each. The rotor stator was rinsed in sterile water to remove residual isopropanol before homogenizing the sponge tissue. The homogenate was poured through a sterile nylon filter (100 μm; autoclaved) to yield a bacterial suspension. We assumed a bacterial density of 8.2×10⁹±7.7×10⁸ cells/mL as previously determined for a Floridian sample of X. muta.¹ The suspension was diluted with 45° C. 1/10 R2Am medium [1.8 g/L R2A Agar (Difco), 12 g/L Nobel agar (Sigma), 33.3 g/L Instant Ocean (pH=7.9)] to the following bacterial densities: 5000, 2500, 1000, 500 and 100 cells/mL. Each diluted bacterial suspension was used to inoculate duplicate ichips as described above. Fully assembled ichips were returned to Runway 10 Reef for implantation in X. muta. A dive knife was used to create slits in sponge tissue adjacent to the site of resection and one ichip was fully inserted into each slit. Coloured pins were placed next to each ichip to indicate its inoculum cell density. Tissue collection, inoculum preparation, ichip assembly and ichip insertion into sponge tissue all occurred within a period of 24 h. The ten ichips were allowed to incubate in the sponge for 7 d before they were removed, packaged into sterile WhirlPak™ plastic bags and transported at ambient temperature to Charlottetown, PE, Canada for microbial domestication.

Microbial domestication. Ichips were processed for microbial domestication according to the published protocol.² All ichips were disassembled aseptically and agar plugs from each through-hole were deposited using a sterile paper clip into separate wells of a 48-well plate (each well contained ˜1 mL of 1/10 R2Am medium). The agar plugs were flattened using the tip of a sterile wooden stick and then incubated for eight weeks at room temperature (˜22° C.) in the dark. After eight weeks, wells that contained morphologically distinct colonies were purified by serial subculturing on 1/10 R2Am. To identify bacterial isolates, a small portion each single colony was first dispersed in 50 □L of DMSO (Sigma). The DMSO cell suspension was used as the template DNA in PCR reactions utilizing the 16S rRNA gene primers pA (5′-AGAGTTTGATCCTGGCTCAG-3′) and pH (5′-AAGGAGGTGATCCAGCC-3′).³ PCR reactions contained EconoTaq PLUS Green 2× master mix (Lucigen) at a 1× concentration, 1 μm □ of each primer and 5% DMSO (v/v) containing suspended cells. Amplicons were directly sequenced using the following primers: 530R (5′-GTATTACCGCGGCTGCTG-3′), 514F (5′-GTGCCAGCASCCGCGG-3′), 936R (5′-GGGGTTATGCCTGAGCAGTTTG-3′) and 1114F (5′-GCAACGAGCGCAACCC-3).^{4,5} Sequences were assembled using Geneious (v7.1). Alteromonas sp. RKMC-009 was most closely related to Alteromonas aestuariivivens JDTF-113T (KY497472) with 98.15% sequence similarity.⁶

The following procedure was carried out to determine the evolutionary relationship of Atleromonas sp. RKMC-009 to all validly described Alteromonas spp., as well as the type representative for all other genera in the Alteromonadaceae. A BlastN search of the GenBank non-redundant nucleotide database (excluding sequences from uncultured/environmental samples) was used to identified previously cultured Alteromonas strains closely related (>99.5% 16S rRNA gene sequence identity) to RKMC-009.⁷ Sequences from strains fitting this criterion were included in the analysis. Pseudoalteromonas haloplanktis (X67024) was used as the outgroup. Phylogenetic analyses were conducted using MEGA X.⁸ Sequences were aligned using the MUSCLE implementation in MEGA X using default parameters.⁹ The alignment was manually corrected and trimmed. The analysis involved 38 nucleotide sequences and a total of 1365 positions. Model testing in MEGA X determined the Kimura 2-parameter model with a Gamma distribution and invariable sites (K2+G+I) model best described the dataset.⁹ Phylogenetic reconstruction was performed using neighbor-joining, unweighted pair group method with arithmetic means, maximum likelihood, maximum parsimony methods.¹⁰ Bootstrap analysis using 1000 replications was used to assess reproducibility of branches in the tree topologies.¹¹

Fermentation and metabolite purification. Seed cultures of Alteromonas sp. RKMC-009 in culture tubes were cultivated overnight in 13 mL of Marine Broth (Difco) at 30° C. with orbital shaking (200 rpm). Seed cultures (30 mL) were used to inoculate ten Fernbach flasks each containing 1 L of BFM4m broth (12 g/L ADM Baker's Soy Flour, 1 g/L NH₄Cl, 12 g/L dextrose, 0.4 g/L agar, 1 g/L CaCO₃, 3 g/L NZ-amine A (Sigma), 18 g/L Instant Ocean, pH=6.8). Fermentations were incubated for 3 d at 30° C. with shaking at 200 rpm. Fermentations were pooled and then extracted 3× with 1.5 L of EtOAc. Pooled EtOAc extracts were dried in vacuo to afford 1.38 g of crude extract. Compound 1 was found to elute between 32.5 min and 35.0 min. Compound 1 was further purified for bioassays by semi-preparative HPLC using a SunFire C₁₈ column (10×250 mm, 5 μm, Waters) with the following elution method [A=H₂O (0.1% formic acid) and B=CH₃CN (0.1% formic acid)]: 90% B from 0 min to 20 min (flow rate=3 mL/min, UV=230, 275 nm).

N-Palmitoyl-α,O-dimethyl-L-tyrosine (1): [α]²⁶_{D}−4.6 (c 0.12, CH₃OH); IR (film) νₘₐₓ 2923, 2852, 1723, 1649, 1613, 1513; ¹H and ¹³C NMR located in Table 2; ESI+HRMS m/z 448.3419 [M+H]⁺ (calcd for C₂₇H₄₆NO₄⁺, 448.3421).

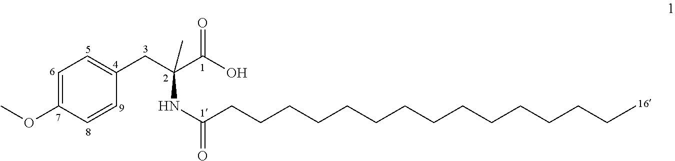 [SMILES: CCCCCCCCCCCCCCCC(=O)N[C@](C)(Cc1ccc(OC)cc1)C(=O)O]

TABLE 2
NMR spectroscopic data (¹H 400 MHz, ¹³C 101 MHz, CDCl₃) of
compound 1.

| Position | | δ_{C}, Type | δ_{H}, mult.(J in Hz) | COSY | HMBC |
|---|---|---|---|---|---|
| 1 | COOH | 177.3, C | 8.61, s(br) | | |
| 2 | | 61.3, C | | | |
| | CH₃ | 23.4, CH₃ | 1.64, s | | 1, 2, 3a/3b |
| | NH | | 6.01, s | | 1, 2, 2-CH₃, 3, 1′ |
| 3a | | 40.3, CH₂ | 3.26, d(13.7) | 3b | 1, 2, 2-CH₃ 4, 5, 9 |
| 3b | | 40.3, CH₂ | 3.39, d(13.7) | 3a | 1, 2, 2-CH₃ 4, 5, 9 |
| 4 | | 128.1, C | | | |
| 5 | | 131.2, CH₂ | 7.05, d(8.6) | 6 | 2, 3, 6, 7, 9 |
| 6 | | 113.9, CH₂ | 6.80, d(8.6) | 5 | 4, 5, 7 |
| 7 | | 158.8, C | | | |
| | OCH₃ | 55.3, CH₃ | 3.77, s | | 7 |
| 8 | | 113.9, CH₂ | 6.80, d(8.6) | 9 | 4, 7, 9 |
| 9 | | 131.2, CH₂ | 7.05, d(8.6) | 8 | 2, 3, 5, 7, 8 |
| 1′ | | 174.1, | | | |
| 2′ | | 37.2, CH₂ | 2.17, t(8.1) | 3′ | 1′, 3′, 4′ |
| 3′ | | 25.7, CH₂ | 1.59, m | 2′, 4′ | 1′, 2′, 4′ |
| 4′ | | 29.4, CH₂ | 1.26, m | | |
| 5′^{a} | | 29.5, CH₂ | 1.26, m | | |
| 6′^{a} | | 29.6, CH₂ | 1.26, m | | |
| 7′^{a} | | 29.8, CH₂ | 1.26, m | | |
| 8′^{a} | | 29.8, CH₂ | 1.26, m | | |
| 9′^{a} | | 29.8, CH₂ | 1.26, m | | |
| 10′^{a} | | 29.8, CH₂ | 1.26, m | | |
| 11′^{a} | | 29.8, CH₂ | 1.26, m | | |
| 12′^{a} | | 29.8, CH₂ | 1.26, m | | |
| 13′^{a} | | 29.8, CH₂ | 1.26, m | | |
| 14′ | | 32.1, CH₂ | 1.25, m | | |
| 15′ | | 22.8, CH₂ | 1.27, m | | |
| 16′ | | 14.3, CH₃ | 0.88, t(6.6) | 15′ | 14′, 15′ |

^{a}Signals for 5′-13′ are interchangeable.

Marfey's analysis. Marfey's method for determination of amino acid configuration was carried out as follows. To separate vials containing dried 1 (5 mg, 0.01 mmol) and O,α-dimethyl-DL-tyrosine (Santa Cruz Biotech, 5 mg, 0.02 mmol) was added 6 M HCl (1 mL), and then the mixtures were heated under reflux overnight with stirring. The reaction mixtures were dried in vacuo and portions of these hydrolysates (1 mg) were transferred to separate vials, to which 150 μL of deionized H₂O, 300 μL of N_{α}-(2,4-dinitro-5-fluorophenyl)-L-alaninamide (L-FDAA; 10 mg/mL in acetone), and 70 μL of aqueous NaHCO₃ (1 M) were added. The reaction mixtures were heated to 37.0° C. for 2 h, quenched with 70 μL of HCl (1 M), and then dried in vacuo. The same process for L-FDAA-derivatization was carried out with α-methyl-L-tyrosine (Sigma-Aldrich; 1 mg, 0.01 mmol). Dried L-FDAA derivatization reactions were suspended in CH₃OH (10 mg/mL) UHPLC-HRMS analysis. These data were acquired on a the following platform: Thermo Accela UHPLC Pump coupled to a Thermo LTQ Orbitrap Velos mass spectrometer fitted with an ESI source and a Thermo Accela PDA. A Kinetex core-shell 100 Å C₁₈ column (2.1×50 mm, 1.7 μm, Phenomenex) was used with a mobile-phase flow rate of 0.5 mL/min and injection volume of 10 μL (samples were prepared in CH₃OH). The following elution method was used [A=H₂O (0.1% formic acid), B=CH₃CN (0.1% formic acid)]: 5% B from 0 to 2 min, linear gradient from 5% B at 2 min to 25% B at 55 min, linear gradient from 25% B at 55 min to 99% B at 57 min, 99% B from 57 to 60 min, linear gradient from 99% B at 60 min to 5% B at 63 min and 5% B from 63 to 70 min. The following HRMS parameters were used: positive ionization mode, mass resolution of 30,000, mass range of m/z 190 to 2,000, spray voltage of 3.4 kV, capillary temperature of 320° C., S-lens RF voltage of 70.0%, maximum injection time of 10 ms, and 1 microscan. The system was controlled by Thermo Xcalibur software modules.

Solvolysis artifact experiment. To determine whether OCH₃-7 in 1 arises from methanolysis, triplicate culture tubes containing 5 mL of BFM4m each were inoculated with overnight seed cultures of RKMC-009 (3% v/v) and incubated with orbital shaking (200 rpm) at 30° C. After 3 d, each culture was extracted with 5 mL of EtOAc. Organic layers from each culture were dried in vacuo and resuspended in CH₃CN (500 μg/mL) for UHPLC-HRMS analysis.

Chemical synthesis. Compounds 1-4 were synthesized using modified literature methodology by N-acylation of the corresponding amino acid with palmitoyl chloride.¹² Compound 3 was dimethylated using a large excess of CH₃I in the presence of Cs₂CO₃ to generate 5 (a synthetic intermediate involved in the preparation of compound 1 (i.e. a methyl ester of compound 1), see Scheme 1 above), which was treated with LiOH to afford 1.¹³ NMR spectra of synthetic compounds 1-5 are located in the Figures.

N-Palmitoyl-O-methyl-L-tyrosine (2). O-Methyl-L-tyrosine (ACROS Organics; 500 mg, 2.56 mmol, 1.00 eq) was stirred with palmitoyl chloride (Alfa Aesar; 7.04 g, 25.61 mmol, 10 eq) in 25 mL of DMF at room temperature. After 16 h, the reaction was diluted with 250 mL of 1N HCl (aq) and extracted with EtOAc (3×100 mL). The combined EtOAc extracts were washed with saturated NaCl (aq) and dried in vacuo. The dried EtOAc extract was resuspended in 250 mL of CH₃CN and extracted with hexanes (5×100 mL). The CH₃CN layer was dried in vacuo and fractionated by automated flash column chromatography using a 25 g silica-pentafluorophenyl column (Silicycle) with the following elution method (A=H₂O and B=CH₃OH): 50% B from 0 to 3 min, linear gradient from 50% B at 3 min to 100% B 25 min and 100% B from 25 to 35 min (flow rate=30 mL/min, UV=230, 275 nm). Compound 2 (1.03 g, 2.38 mmol, 93%) was obtained as an amorphous white solid: [α]²⁶_{D}17.1 (c 0.46, CH₃OH); IR (film) νₘₐₓ 3296, 2919, 2850, 1730, 1706, 1642, 1614, 1534, 1514 cm⁻¹; ¹H NMR (CDCl₃, 400 MHz) 8.29 (1H, s, COOH), 7.07 (2H, d, J=8.6 Hz, H-5, H-9), 6.83 (2H, d, J=8.6 Hz, H-6, H-8), 5.97 (1H, d, J=7.4 Hz, CONH), 4.82 (1H, dt, J=7.4, 5.9 Hz, H-2), 3.78 (3H, s, OCH₃), 3.17 (1H, dd, J=14.2, 5.8 Hz, H-3b), 3.07 (1H, dd, J=14.2, 5.8 Hz, H-3a), 2.18 (2H, td, J=7.7, 1.7 Hz, H-2′), 1.56 (2H, m, H-3′), 1.21-1.33 (24H, m, H-4′, H-5′, H-6′, H-7′, H-8′, H-9′, H-10′, H-11′, H-12′, H-13′, H-14′, H-15′), 0.88 (3H, t, J=7.0 Hz, H-16′); ¹³C NMR (CDCl₃, 151 MHz) 175.09 (C, C-1), 174.1 (C, C-1′), 158.9 (C, C-7), 130.5 (CH, C-5, C-9), 127.7 (C, C-4), 114.2 (CH, C-6, C-8), 55.3 (CH₃, OCH₃), 53.5 (CH, C-2), 36.6 (CH2, C-2′), 36.5 (CH2, C-3), 32.1 (CH2, C-14′), 29.8 (CH₂), 29.8 (CH₂), 29.8 (CH₂), 29.8 (CH₂), 29.8 (CH₂), 29.8 (CH₂), 29.6 (CH₂), 29.5 (CH₂), 29.5 (CH₂), 29.3 (CH₂, C-4′), 25.7 (CH₂, C-₃′), 22.8 (CH2, C-15′), 14.3 (CH₃, C-16′); ESI+HRMS m/z 434.3262 [M+H]⁺ (calcd for C₂₆H₄₄NO₄⁺, 434.3265).

N-Palmitoyl-α-methyl-L-tyrosine (3). α-Methyl-L-tyrosine (Sigma-Aldrich; 200 mg, 1.02 mmol, 1.00 eq) was stirred with palmitoyl chloride (Alfa Aesar; 2.82 g, 10.26 mmol, 10 eq) in 10 mL of DMF at room temperature. After 16 h, the reaction was diluted with 100 mL of 1N HCl (aq) and extracted with EtOAc (3×100 mL). The combined EtOAc extracts were washed with saturated NaCl (aq) and dried in vacuo. The dried EtOAc extract was resuspended in 250 mL of CH₃CN and extracted with hexanes (5×100 mL). The CH₃CN layer was dried in vacuo and fractionated using automated flash column chromatography (identical conditions as for 2) to afford 3 (394 mg, 0.91 mmol, 89%) as an amorphous white solid: [α]²⁶_{D}−18.1 (c 0.20, CH₃OH); IR (film) νₘₐₓ 3326, 2923, 2853, 2156, 1716, 1646, 1615, 1516 cm⁻¹; ¹H NMR (CDCl₃, 400 MHz) 6.95 (2H, d, J=8.6 Hz, H-5, H-9), 6.72 (2H, d, J=8.6 Hz, H-6, H-8), 6.10 (1H, s, CONH), 3.38 (1H, d, J=13.7 Hz, H-3b), 3.17 (1H, d, J=13.7 Hz, H-3a), 2.17 (2H, t, 7.9 Hz, C-2′), 1.63 (3H, s, CH₃-2), 1.58 (2H, m, H-3′), 1.21-1.32 (24H, m, H-4′, H-5′, H-6′, H-7′, H-8′, H-9′, H-10′, H-11′, H-12′, H-13′, H-14′, H-15′), 0.88 (3H, t, J=6.7 Hz, H-16′); ¹³C NMR (CDCl₃, 151 MHz) 177.7 (C, C-1), 174.5 (C, C-1′), 155.1 (C, C-7), 131.3 (CH, C-5, C-9), 127.6 (C, C-4), 115.5 (CH, C-6, C-8), 61.5 (C, C-2), 40.6 (CH₂, C-3), 37.3 (CH2, C-2′), 32.1 (CH₂, C-14′), 29.9 (CH₂), 29.9 (CH₂), 29.9 (CH₂), 29.8 (CH₂), 29.8 (CH₂), 29.8 (CH₂), 29.7 (CH₂), 29.5 (CH₂), 29.5 (CH₂), 29.4 (CH₂, C-4′), 25.7 (CH₂, C-3′), 23.3 (CH₂, CH₃-2), 22.8 (CH₂, C-15′), 14.3 (CH₃, C-16′); ESI+HRMS m/z 434.3260 [M+H]⁺ (calcd for C₂₆H₄₄NO₄⁺, 434.3265).

N-Palmitoyl-L-tyrosine (4). L-Tyrosine (AMRESCO; 500 mg, 2.76 mmol, 1.00 eq) was stirred with palmitoyl chloride (Alfa Aesar; 7.59 g, 27.60 mmol, 10 eq) in 25 mL of DMF at room temperature. After 16 h, the reaction was diluted with 250 mL of 1N HCl (aq) and extracted with EtOAc (3×100 mL). The combined EtOAc extracts were washed with saturated NaCl (aq) and dried in vacuo. The dried EtOAc extract was resuspended in 250 mL of CH₃CN and extracted with hexanes (5×100 mL). The CH₃CN layer was dried in vacuo and fractionated using automated flash column chromatography (identical conditions as for 2) to afford 4 (1.09 g, 2.59 mmol, 94%): [α]²⁶_{D}+17.4 (c 0.26, CH₃OH); IR (film) νₘₐₓ 3312, 3234, 2915, 2848, 2516, 1705, 1643, 1541, 1516 cm⁻¹; ¹H NMR (CDCl₃, 400 MHz) 12.54 (1H, s, COOH), 9.16 (1H, s, OH-7), 7.99 (1H, d, J=8.2 Hz, CONH), 6.99 (2H, d, J=8.5 Hz, H-5, H-9), 6.63 (2H, d, J=8.5 Hz, H-6, H-8), 4.32 (1H, m, H-2), 2.90 (1H, dd, J=14.1, 4.8 Hz, H-3b), 2.71 (1H, dd, J=14.1, 9.6 Hz, H-3a), 2.03 (2H, t, J=7.4 Hz, H-2′), 1.39 (2H, m, H-3′), 1.14-1.24 (24H, m, H-4′, H-5′, H-6′, H-7′, H-8′, H-9′, H-10′, H-11′, H-12′, H-13′, H-14′, H-15′), 0.85 (3H, t, J=7.0 Hz, H-16′); ¹³C NMR (CDCl₃, 151 MHz) 173.4 (C, C-1), 172.1 (C, C-1′), 155.9 (C, C-7), 127.7 (C, C-4), 130.0 (CH, C-5, C-9), 114.9 (CH, C-6, C-8), 53.6 (CH, C-2), 36.0 (CH₂, C-2′), 35.1 (CH₂, C-3), 31.3 (CH₂, C-14′), 29.1 (CH₂), 29.1 (CH₂), 29.1 (CH₂), 29.1 (CH₂), 29.1 (CH₂), 29.0 (CH₂), 28.9 (CH₂), 28.8 (CH₂), 28.7 (CH₂), 28.5 (CH2, C-4′), 25.2 (CH2, C-3′), 22.1 (CH₂, C-15′), 14.0 (CH₃, C-16′); ESI+HRMS m/z 420.3091 [M+H]⁺ (calcd for C₂₅H₄₂NO₄⁺, 420.3108).

N-Palmitoyl-α,O-dimethyl-L-tyrosine methyl ester (5). To a solution of 3 (75 mg, 0.17 mmol, 1.00 eq) in DMSO (5 mL) was added Cs₂CO₃ (Sigma-Aldrich; 118 mg, 0.36 mmol, 2.10 eq) and CH₃I (Sigma-Aldrich; 1.06 mL, 17.30 mmol, 100.00 eq), which were stirred for 16 h at room temperature. The reaction was diluted with 10 mL of H₂O and extracted with CHCl₃ (3×50 mL). The combined CHCl₃ extracts were washed with saturated NaCl (aq) and dried in vacuo. The dried CHCl₃ extract was fractionated using automated flash column chromatography (identical conditions as for 2) to afford 5 (21 mg, 0.05 mmol, 27%): ¹HNMR (CDCl₃, 400 MHz) δ6.95 (2H, d, J=8.7 Hz), 6.79 (2H, d, J=8.7 Hz), 6.01 (1H, s), 3.77 (3H, s), 3.77 (3H, s), 3.49 (1H, d, J=13.5 Hz), 3.13 (1H, d, J=13.5 Hz), 2.13 (2H, t, J=7.09 Hz), 16.4 (3H, s), 1.60 (1H, m), 1.23-1.31 (24H, m), 0.89 (3H, t, J=7.0 Hz); ¹³C NMR (CDCl₃, 151 MHz) δ174.8, 172.6, 158.7, 131.0, 128.6, 113.8, 61.3, 55.3, 52.7, 40.6, 37.4, 32.1, 29.8, 29.8, 29.8, 29.8, 29.8, 29.8, 29.7, 29.5, 29.5, 29.4, 25.7, 23.4, 22.8, 14.3. ESI+HRMS m/z 462.3582 [M+H]⁺ (calcd for C₂₈H₄₈NO₄⁺, 462.3578).

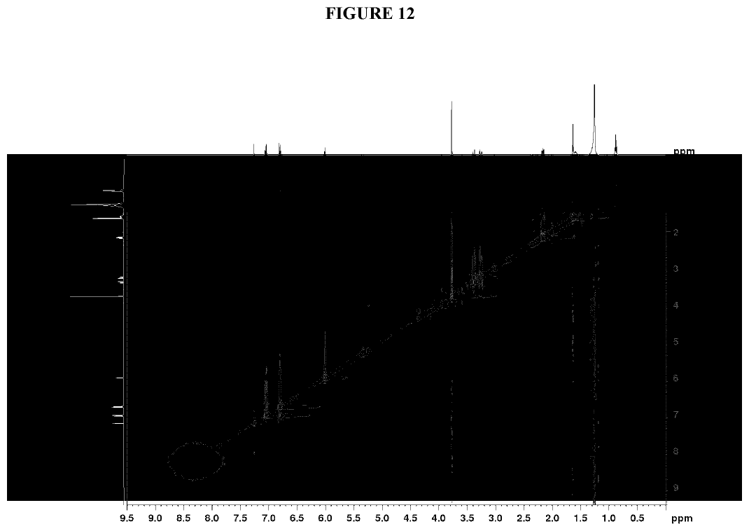

N-Palmitoyl-α,O-dimethyl-L-tyrosine (1). To a solution of 5 (10 mg, 0.02 mmol, 1.00 eq) in 75% THF (aq) (1 mL) was added LiOH (aq) (Sigma-Aldrich; 100 μL of 10 mg/mL solution, 0.04 mmol, 2.00 eq), which was stirred for 48 h at room temperature. The reaction was diluted with 10 mL of H₂O and extracted with CHCl₃ (3×50 mL). The combined CHCl₃ extracts were washed with saturated NaCl (aq) and dried in vacuo. The dried CHCl₃ extract was fractionated using automated flash column chromatography (identical conditions as for 2) to afford 1 (9 mg, 0.02 mmol, 95%): [α]²⁶_{D}−5.1 (c 0.15, CH₃OH); IR (film) νₘₐₓ 2923, 2852, 1723, 1649, 1613, 1513; ¹H and ¹³C NMR are located in FIGS. 12-13; ESI+HRMS m/z 448.3425 [M+H]⁺ (calcd for C₂₇H₄₆NO₄⁺, 448.3421).

Evaluation of antimicrobial activity and cytotoxicity. The following five Enterococcus spp. were isolated from clinical specimens at the Atlantic Veterinary College (AVC) by the AVC Diagnostic Services Bacteriology Laboratory. Isolates were identified using the Bruker microflex LT MALDI-TOF with MBT Compass version 4.179. The direct colony transfer method was used with the Bruker Matrix HCCA (α-cyano-4-hydroxycinnamic acid) overlay, following manufacturer guidelines. Score values between 2.00 and 3.00 were considered high confidence identifications. The following isolates (with source species) were identified: E. faecium 15337 (feline), E. faecalis 16371 (canine), E. gallinarum 20993 (erinaceine), E. casseliflavus 15984 (equine) and E. hirae 17446 (avine). Antimicrobial activity of 1-4 was evaluated against all five clinical Enterococcus isolates in addition to methicillin-resistant Staphylococcus aureus ATCC 33591 (MRSA), S. warneri ATCC 17917, vancomycin-resistant Enterococcus faecium EF379 (VRE), Pseudomonas aeruginosa ATCC 14210, Proteus vulgaris ATCC 12454 and Candida albicans ATCC 14035. All testing was carried out in triplicate according to the Clinical Laboratory Standards Institute testing standards in a 96-well plate microbroth dilution assay as previously described.¹⁴ Optical density was measured using a Thermo Scientific Varioskan Flash plate reader at 600 nm, recording at time zero and then again after incubation for 22 h (37° C.) to determine percent growth inhibition. Cytotoxicity was evaluated against Vero kidney cell line from African green monkey (Cercopithecus aethiops), MCF7 human breast adenocarcinoma cells (ATCC HTB-22), human breast adenocarcinoma cells (ATCC HTB-26) and HCT-116 human colorectal carcinoma cells (ATCC CCL-247). All assays were carried out as described previously.¹⁴ Fluorescence was measured using a Thermo Scientific Varioskan Flash plate reader at 560/12 excitation, 590 nm emission both at time zero and 4 h after alamarBlue (Invitrogen) addition. For both antimicrobial and cytotoxicity data, growth inhibition was expressed as a percentage and plotted against the logarithm of concentration. Four-parameter dose-response curves were fit to these data using the variable slope model in GraphPad Prism 8.0.2.

One or more illustrative embodiments have been described by way of example. It will be understood to persons skilled in the art that a number of variations and modifications can be made without departing from the scope of the invention as defined in the claims.

### REFERENCES

(1) Rappé, M. S.; Giovannoni, S. J. Annu. Rev. Microbiol. 2003, 57 (1), 369.

(2) Schloss, P. D.; Handelsman, J. Microbiology and Molecular Biology Reviews 2004, 68 (4), 686.

(3) Kaeberlein, T.; Lewis, K.; Epstein, S. S. Science 2002, 296 (5570), 1127.

(4) Nichols, D.; Cahoon, N.; Trakhtenberg, E. M.; Pham, L.; Mehta, A.; Belanger, A.; Kanigan, T.; Lewis, K.; Epstein, S. S. Appl. Environ. Microbiol. 2010, 76 (8), 2445.

(5) Ling, L. L.; Schneider, T.; Peoples, A. J.; Spoering, A. L.; Engels, I.; Conlon, B. P.; Mueller, A.; Schaberle, T. F.; Hughes, D. E.; Epstein, S.; Jones, M.; Lazarides, L.; Steadman, V. A.; Cohen, D. R.; Felix, C. R.; Fetterman, K. A.; Millett, W. P.; Nitti, A. G.; Zullo, A. M.; Chen, C.; Lewis, K. Nature 2015, 517 (7535), 455.

(6) Gloeckner, V.; Wehrl, M.; Moitinho-Silva, L.; Gernert, C.; Schupp, P.; Pawlik, J. R.; Lindquist, N. L.; Erpenbeck, D.; Worheide, G.; Hentschel, U. Biol. Bull. 2014, 227 (1), 78.

(7) Hentschel, U.; Usher, K. M.; Taylor, M. W. FEMS Microbi-ol. Ecol. 2006, 55 (2), 167.

(8) Maclntyre, L. W.; Haltli, B. A.; Kerr, R. G. Microbiology Re-source Announcements 2019, 8 (25).

(9) The European Committee on Antimicrobial Susceptibility Testing. Breakpoint tables for interpretation of MICs and zone diameters. Version 9.0, 2019. http://www.eucast.org.

(10) Bruns, H.; Thiel, V.; Voget, S.; Patzelt, D.; Daniel, R.; Wagner Dobler, I.; Schulz, S. Chemistry & Biodiversity 2013, 10 (9), 1559.

(11) Bruns, H.; Herrmann, J.; Müll, R.; Wang, H.; Wagner Dobler, I.; Shulz, S. J. Nat. Prod. 2018, 81 (1), 131.

(12) Bruns, H.; Ziesche, L.; Taniwal, N. K.; Wolter, L.; Brink-hoff, T.; Herrmann, J.; Müller, R.; Schulz, S. Beilstein J Org Chem 2018, 14, 2964.

(13) Brady, S. F.; Chao, C. J.; Clardy, J. J. Am. Chem. Soc. 2002, 124 (34), 9968.

(14) Deering, R. W.; Chen, J.; Sun, J.; Ma, H.; Dubert, J.; Barja, J. L.; Seeram, N. P.; Wang, H.; Rowley, D. C. J. Nat. Prod. 2016, 79 (2), 447.

(15) Puk, O.; Bischoff, D.; Kittel, C.; Pelzer, S.; Weist, S.; Steg-mann, E.; Süssmuth, R. D.; Wohlleben, W. Journal of Bacteriolo-gy 2004, 186 (18), 6093.

(16) Heide, L. Nat Prod Rep 2009, 26 (10), 1241.

(17) Zhang, S.; Zhu, J.; Zechel, D. L.; Jessen-Trefzer, C.; East-man, R. T.; Paululat, T.; Bechthold, A. ChemBioChem 2017, 19 (3), 272.

(18) Liu, J.; Wang, B.; Li, H.; Xie, Y.; Li, Q.; Qin, X.; Zhang, X.; Ju, J. Org. Lett. 2015, 17 (6), 1509.

¹Gloeckner, V.; Wehrl, M.; Moitinho-Silva, L.; Gernert, C.; Schupp, P.; Pawlik, J. R.; Lindquist, N. L.; Erpenbeck, D.; Worheide, G.; Hentschel, U. Biol. Bull. 2014, 227 (1), 78-88.

²Nichols, D.; Cahoon, N.; Trakhtenberg, E. M.; Pham, L.; Mehta, A.; Belanger, A.; Kanigan, T.; Lewis, K.; Epstein, S. S. Appl. Environ. Microbiol. 2010, 76 (8), 2445-2450.

³Edwards, U.; Rogall, T.; Blocker, H.; Emde, M.; Bottger, E. C. Nucleic Acids Res 1989, 17 (19), 7843-7853.

⁴Manefield, M.; Whiteley, A. S.; Griffiths, R. I.; Bailey, M. J. Appl. Environ. Microbiol. 2002, 68 (11), 5367-5373.

⁵Gontang, E. A.; Fenical, W.; Jensen, P. R. Appl. Environ. Microbiol. 2007, 73 (10), 3272-3282.

⁶MacIntyre, L. W.; Haltli, B. A.; Kerr, R. G. Microbiology Resource Announcements 2019, 8 (25).

⁷Zhang, Z.; Schwartz, S.; Wagner, L.; Miller, W. J. Comput. Biol. 2000, 7 (1-2), 203.

⁸Kumar, S.; Stecher, G.; Li, M.; Knyaz, C.; Tamura, K. Mol. Biol. Evol. 2018, 35 (6), 1547.

⁹Edgar, R. C. Nucleic Acids Res 2004, 32 (5), 1792.

¹⁰Saitou, N.; Nei, M. Mol. Biol. Evol. 1987, 4 (4), 406.

¹¹Felsenstein, J. Evolution 1985, 39 (4), 783.

¹²Brady, S. F.; Clardy, J. J. Am. Chem. Soc. 2000, 122 (51), 12903-12904.

¹³Burch, P.; Chicca, A.; Gertsch, J.; Gademann, K. ACS Med. Chem. Lett. 2014, 5 (2), 172-177.

¹⁴Overy, D. P.; Berrue, F.; Correa, H.; Hanif, N.; Hay, K.; Lanteigne, M.; McQuillan, K.; Duffy, S.; Boland, P.; Jagannathan, R.; Carr, G. S.; Vansteeland, M.; Kerr, R. G. Mycology 2014, 5 (3), 130-144.

Maclntyre, L. W, Haltli, B. A., Kerr, R. G., Microbiology Resource Announcements (2019), 8(25): 2445

All references cited herein and elsewhere in the specification are herein incorporated by reference in their entireties.

1. A compound of formula A: 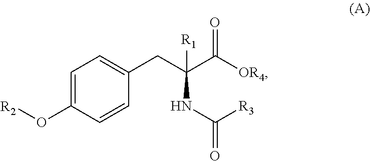
wherein
R₁ is —H; or a linear or branched, optionally substituted, C₁-C₆ alkyl; or optionally substituted phenyl;
R₂ is —H; or a linear or branched, optionally substituted, C₁-C₆ alkyl; or optionally substituted phenyl;
R₃ is linear or branched, optionally substituted, C₅-C₂₀ alkyl, alkenyl, or alkynyl; and
R₄ is —H; linear or branched, optionally substituted, C₁-C₆ alkyl; or optionally substituted phenyl;
or a prodrug, ester, or pharmaceutically acceptable salt or solvate thereof.

2. The compound of claim 1, wherein R₁ is —H, Me, Et, nPr, iPr, tBu, iBu, secBu, nBu, or Ph and wherein R₂ is —H, Me, Et, nPr, iPr, tBu, iBu, secBu, nBu, or Ph.

3. (canceled)

4. The compound of claim 1, wherein R₃ is a C₅-C₂₀ group with 0-3 double and/or triple carbon-carbon bonds (0-3 Δ).

5. (canceled)

6. (canceled)

7. The compound of claim 1, having formula B: 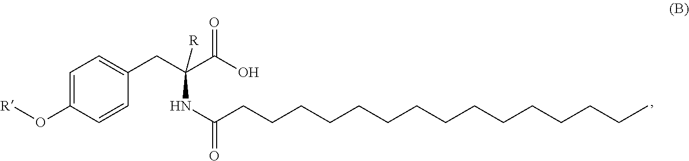
wherein
R is —CH₃ or —H; and
R′ is —CH₃ or —H;
or a prodrug, ester, or pharmaceutically acceptable salt or solvate thereof.

8. The compound of claim 1, having formula 1, 2, 3 or 4 as follows: 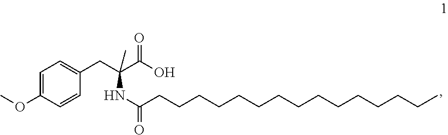 [SMILES: CCCCCCCCCCCCCCCC(=O)N[C@](C)(Cc1ccc(OC)cc1)C(=O)O]
or a prodrug, ester, or pharmaceutically acceptable salt or solvate thereof; 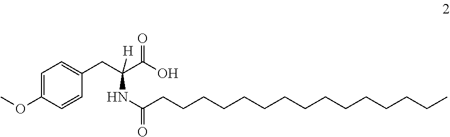 [SMILES: CCCCCCCCCCCCCCCC(=O)N[C@H](Cc1ccc(OC)cc1)C(=O)O]
or a prodrug, ester, or pharmaceutically acceptable salt or solvate thereof; 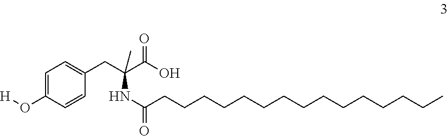 [SMILES: CCCCCCCCCCCCCCCC(=O)N[C@](C)(Cc1ccc(O)cc1)C(=O)O]
or a prodrug, ester, or pharmaceutically acceptable salt or solvate thereof; 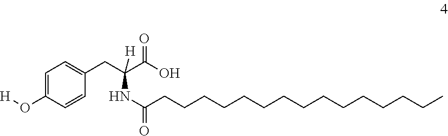 [SMILES: CCCCCCCCCCCCCCCC(=O)N[C@H](Cc1ccc(O)cc1)C(=O)O]
or a prodrug, ester, or pharmaceutically acceptable salt or solvate thereof.

9. (canceled)

10. (canceled)

11. (canceled)

12. A pharmaceutical composition comprising the compound of claim 1, and a pharmaceutically acceptable excipient or carrier.

13. (canceled)

14. (canceled)

15. A method for reducing or preventing growth of a bacteria, said method comprising:
contacting the bacteria with the compound of claim 1.

16. A method for treating or preventing a bacterial infection in a subject in need thereof, said method comprising:
administering the compound of claim 1 to the subject.

17. The method of claim 16, wherein the bacteria comprises a Staphylococcus or Enterococcus bacteria.

18. The method of claim 16, wherein the bacteria comprises Staphylococcus aureus, Staphylococcus warneri, or Enterococcus faecium.

19. (canceled)

20. (canceled)

21. The method of claim 16, wherein the bacteria comprises an Enterococcus and the compound comprises formula 1 or 3 as follows: 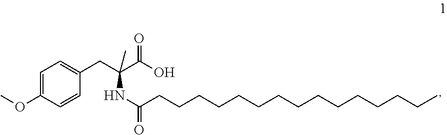 [SMILES: CCCCCCCCCCCCCCCC(=O)N[C@](C)(Cc1ccc(OC)cc1)C(=O)O]
or a prodrug, ester, or pharmaceutically acceptable salt or solvate thereof; 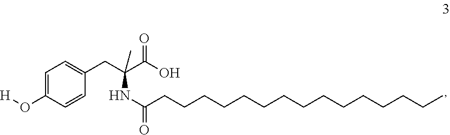 [SMILES: CCCCCCCCCCCCCCCC(=O)N[C@](C)(Cc1ccc(O)cc1)C(=O)O]
or a prodrug, ester, or pharmaceutically acceptable salt or solvate thereof.

22. (canceled)

23. The method of claim 16, wherein the bacteria comprises S. aureus, and the compound comprises formula 1 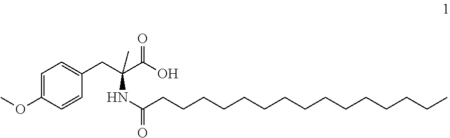 [SMILES: CCCCCCCCCCCCCCCC(=O)N[C@](C)(Cc1ccc(OC)cc1)C(=O)O]
or a prodrug, ester, or pharmaceutically acceptable salt or solvate thereof;
formula 3 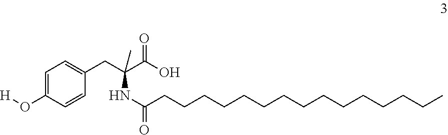 [SMILES: CCCCCCCCCCCCCCCC(=O)N[C@](C)(Cc1ccc(O)cc1)C(=O)O]
or a prodrug, ester, or pharmaceutically acceptable salt or solvate thereof;
or both.

24. The method of claim 16, wherein:
the bacteria comprises an Enterococcus or Staphylococcus and the compound comprises an α-methyl substituent (R₁═CH₃) or
the bacteria comprises an Enterococcus and the compound comprises an O-methylation at the tyrosine side chain (R₂═—CH₃) or
the bacteria comprises a Staphylococcus and the compound comprises a tyrosine side chain (R₂═—H).

25. (canceled)

26. (canceled)

27. A bacterial sample comprising Alteromonas RKMC-009.

28. A method for producing a compound as defined in claim 1, said method comprising:
providing a compound of formula C: 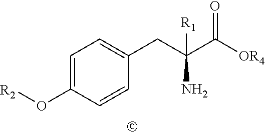
wherein
R₁ is —H; or a linear or branched, optionally substituted, C₁-C₆ alkyl; or optionally substituted phenyl;
R₂ is —H; or a linear or branched, optionally substituted, C₁-C₆ alkyl; or optionally substituted phenyl;
R₄ is —H; linear or branched, optionally substituted, C₁-C₆ alkyl; or optionally substituted phenyl; and
performing an N-acylation with a linear or branched, optionally substituted, C₆-C₂₁ acyl chloride.

29. The method of claim 28, wherein the compound comprises formula 1, 2, 3 or 4 as follows: 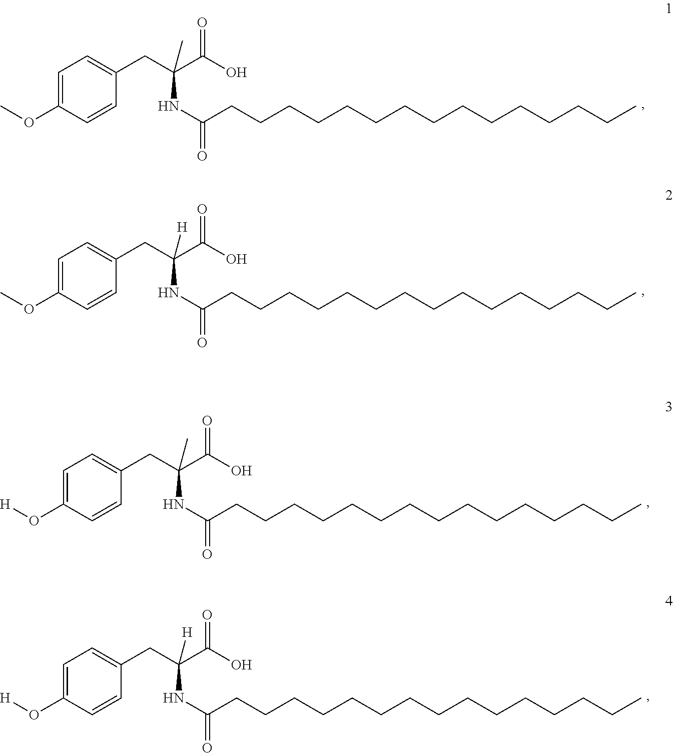
and the method comprises reacting O-methyl-α-methyl-L-tyrosine with palmitoyl chloride for N-acylation.

30. (canceled)

31. (canceled)

32. (canceled)

33. (canceled)

34. A method for producing a compound of formula 1, 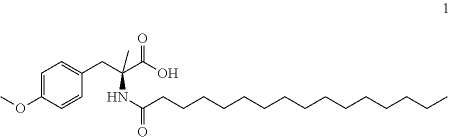 [SMILES: CCCCCCCCCCCCCCCC(=O)N[C@](C)(Cc1ccc(OC)cc1)C(=O)O]
said method comprising:
fermenting Alteromonas sp. RKMC-009 bacteria in BFM4m broth; and
extracting the broth with EtOAc.

35. (canceled)

36. The method of claim 34, wherein the Alteromonas sp. RKMC-009 bacteria is Alteromonas RKMC-009 deposited under NRRL accession number NRRL B-67979, or a functional equivalent thereof.

37. A composition or bacterial sample comprising Alteromonas RKMC-009 deposited under NRRL accession number NRRL B-67979, or a functional equivalent thereof.

38. A lysate, supernatant, broth, or extract derived or prepared from an Alteromonas sp. RKMC-009 bacterial culture or ferment, wherein the lysate, supernatant, broth, or extract comprises a compound of formula 1: 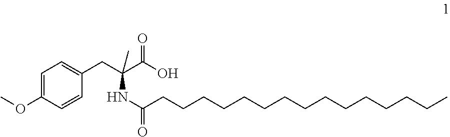 [SMILES: CCCCCCCCCCCCCCCC(=O)N[C@](C)(Cc1ccc(OC)cc1)C(=O)O]
or a salt thereof.

39. The lysate, supernatant, broth, or extract of claim 38, wherein the Alteromonas sp. RKMC-009 bacterial culture or ferment comprises Alteromonas sp. RKMC-009 deposited under NRRL accession number NRRL B-67979, or a functional equivalent thereof.
